SEQUENCE LISTING (3 sequences)

SEQ ID NO: 1 - DNA, 19 bp, synthetic construct
  source 1..19: mol_type = other DNA
taaagatgtatcctatatc

SEQ ID NO: 2 - DNA, 19 bp, synthetic construct
  source 1..19: mol_type = other DNA
ttaactgaaatgaaatcac

SEQ ID NO: 3 - DNA, 19 bp, synthetic construct
  source 1..19: mol_type = other DNA
taagttcacatgtcctttc

(19) United States
(12) Patent Application Publication
(10) Pub. No.: US 2026/0258420 A1
(43) Pub. Date: Sep. 3, 2026

(54) COMPOSITIONS AND METHODS FOR THE TREATMENT OF HUMAN PAPILLOMAVIRUS-DRIVEN CANCERS

(71) Applicant: Board of Regents, The University of Texas System, Austin, TX (US)

(72) Inventors: Soma Ghosh, Houston, TX (US); Faye M. Johnson, Houston, TX (US)

(73) Assignee: Board of Regents, The University of Texas System, Austin, TX (US)

(21) Appl. No.: 19/162,791

(22) PCT Filed: Mar. 6, 2024
(86) PCT No.: PCT/US2024/018643

§ 371 Date: Sep. 5, 2025

Related U.S. Application Data
(60) Provisional application No. 63/489,141, filed on Mar. 8, 2023.

(51) Int. Cl. C12N 15/113; A61K 9/00; A61K 31/4439; A61K 31/55; A61K 45/06; A61P 35/00

(52) U.S. Cl. CPC C12N 15/1136; A61K 9/0053; A61K 31/4439; A61K 31/55; A61K 45/06; A61P 35/00; C12N 2310/14

(57) ABSTRACT

The present invention provides methods and compositions for treating HPV-driven cancers in a subject comprising the use of therapeutic compounds to reduce Aurora kinase function. The present invention also provides methods and compositions for treating HPV-driven cancers in a subject comprising the use of therapeutic compounds to reduce Aurora kinase function in combination with therapeutic compounds to reduce TRIP13 function.

### CROSS-REFERENCE TO RELATED APPLICATIONS

This application claims the priority of U.S. Provisional Appl. Ser. No. 63/489,141, filed Mar. 8, 2023, the entire disclosure of which is incorporated herein by reference.

### STATEMENT OF GOVERNMENT RIGHTS

This invention was made with government support under CA248205 awarded by the National Institutes of Health. The government has certain rights in the invention.

### INCORPORATION OF SEQUENCE LISTING

A sequence listing containing the file named “MDCC009WO_ST26” which is 3.48 kilobytes (measured in MS-Windows®) and created on Feb. 28, 2024, and comprises 3 sequences, is incorporated herein by reference in its entirety.

### FIELD OF THE INVENTION

The present disclosure relates to the field of cancer therapy, and more specifically to compositions and methods for the treatment of human papillomavirus (HPV)-driven cancers.

### BACKGROUND OF THE INVENTION

Each year, there are 694,000 new cases of human papillomavirus (HPV)-driven cancers worldwide. HPV causes more than 5% of cancers, including head and neck squamous cell carcinoma (HNSCC) and squamous cancers of the cervix, anus, penis, and vulva. HPV-driven cervical cancer is the leading cause of cancer death in women in the developing world, and the incidence of HPV-positive HNSCC in the developed world is rising. The treatment of HPV-positive HNSCC, which usually presents as locally advanced disease, involves a combination of cisplatin chemotherapy and radiotherapy and results in decades-long, chronic, therapy-related adverse effects, including difficulty swallowing, dry mouth, feeding tube dependence, and aspiration pneumonia. Consequently, de-intensified therapies and new therapies focusing on druggable targets for HPV-associated HNSCC are needed to reduce mortality and treatment-associated morbidity.

Several independent investigators have defined the genetic landscape of HPV-positive HNSCC, which is distinct from HPV-negative HNSCC and have identified potential pathways controlling cell cycle dysregulation and constitutive cell proliferation (Nature 2015; 517(7536):576-82; Gillison, et al., Genome Res 2019; 29(1):1-17). Another distinct molecular feature of HPV-positive cancers is that they express the viral oncoproteins E6 and E7, which lead to p53 and retinoblastoma (Rb) protein degradation and thus disrupts cell cycle regulation and provides a mechanism for tumorigenesis. None of these findings, however, has translated into improved therapies.

Although HPV-positive tumors are molecularly distinct from HPV-negative tumors, their treatments are identical. The present disclose, provides the identification of HPV status-selective therapies using a high-throughput drug screen (HTDS) with diverse drugs. The present disclosure focused on identifying drugs that caused cell death and identified Aurora kinase inhibitors as the only drug class that was consistently more effective against HPV-positive cancers than HPV-negative cancers. The present disclosure demonstrates that Rb-deficient, HPV-positive cells that overexpress the mitotic checkpoint gene Mad2 (MAD2L1) rely on both thyroid hormone receptor interaction 13 (TRIP13) and Aurora kinase activity to maintain mitotic fidelity, such that the combined inhibition of TRIP13 and Aurora kinase activity can lead to irreversible mitotic arrest, DNA damage, and apoptosis. Therefore, the present disclosure demonstrates the roles of Rb, Mad2, and TRIP13 in Aurora kinase inhibition-induced apoptosis in HPV-positive cancers. The present disclosure further demonstrates that Rb-deficient cancers are sensitive to Aurora kinase inhibition because of an imbalance between Mad2 and TRIP13. The present disclosure demonstrates for the first time that TRIP13 depletion in combination with Aurora kinase inhibition leads to significantly more apoptosis than does single pathway inhibition selectively in HPV-positive cancers.

### SUMMARY OF THE INVENTION

In one aspect, the present disclosure provides a method of treating HPV-positive cancer in a subject in need thereof, the method comprising administering a therapeutically effective amount of an Aurora kinase inhibitor to said subject. In one embodiment, the HPV-positive cancer is selected from the group consisting of head and neck squamous cell carcinoma, cervical squamous cell carcinoma, anal squamous cell carcinoma, penile squamous cell carcinoma, and vulvar squamous cell carcinoma. In another embodiment, the Aurora kinase inhibitor is selected from the group consisting of alisertib, CYC116, tozasertib, ZM447439, GSK1070916, PF03814735, SNS314, AMG900, MLN8054, CCT129202, CCT137690, MK5108, PHA680632, danusertib, TAK901, barasertib, ENMD-2076, AT-9283, KW-2449, ilorasertib, chiauranib, LY3295668 (AK-01), and TT-00420, or a combination thereof. In yet another embodiment, the administering comprises local, regional, systemic, or continual administration. In still yet another embodiment, the administering comprises providing a single dose. The administering, in one embodiment, comprises providing multiple doses. In another embodiment, the administering comprises oral, intravenous, or intramuscular administration. The administering, in yet another embodiment, comprises providing a pharmaceutical composition comprising the Aurora kinase inhibitor to the subject.

In another aspect, the present disclosure provides a method further comprising administering a second therapy to said subject. In one embodiment, the second therapy is selected from the group consisting of an immunotherapy, a targeted cancer therapy, a chemotherapy, a radiation therapy, and surgery. Administering the second therapy, in another embodiment, comprises administering a therapeutically effective amount of a therapeutic composition effective to decrease the activity of TRIP13 in the subject. In yet another embodiment, the therapeutic composition comprises a protein, a peptide, a polypeptide, an RNA molecule, a peptidomimetic, an siRNA molecule, a gRNA molecule, or a small molecule, and said therapeutic composition results in a reduction of TRIP13 activity in a cancer cell of the subject. The therapeutic composition, in still yet another embodiment, performs a function selected from the group consisting of: (a) reducing or inhibiting the expression of a gene encoding TRIP13; (b) reducing or inhibiting the expression of a TRIP13 protein; and (c) reducing or inhibiting the function of a TRIP13 protein. In one embodiment, the therapeutic composition comprises an siRNA molecule complementary to at least a portion of an mRNA sequence encoded by the TRIP13 gene, or a gRNA molecule complementary to at least a portion of the TRIP13 gene. In another embodiment, the therapeutic composition comprises DCZ0415. The second therapy, in yet another embodiment, is administered prior to administering the Aurora kinase inhibitor. In still yet another embodiment the second therapy is administered after administering the Aurora kinase inhibitor. In one embodiment, the second therapy is administered approximately simultaneously with the administration of the Aurora kinase inhibitor. Administering the second therapy, in another embodiment, comprises local, regional, systemic, or continual administration. Administering the second therapy, in yet another embodiment, comprises providing a single dose. Administering the second therapy, in still yet another embodiment, comprises providing multiple doses. Administering the second therapy comprises, in one embodiment, oral, intravenous, or intramuscular administration.

In yet another aspect, the present disclosure provides a pharmaceutical composition comprising: (a) an Aurora kinase inhibitor selected from the group consisting of alisertib, CYC116, tozasertib, ZM447439, GSK1070916, PF03814735, SNS314, AMG900, MLN8054, CCT129202, CCT137690, MK5108, PHA680632, danusertib, TAK901, barasertib, ENMD-2076, AT-9283, KW-2449, ilorasertib, chiauranib, LY3295668 (AK-01), and TT-00420; and (b) DCZ0415, an siRNA molecule complementary to at least a portion of an mRNA sequence encoded by the TRIP13 gene, a gRNA molecule complementary to at least a portion of the TRIP13 gene, or a chemotherapeutic agent. In one embodiment, the pharmaceutical composition further comprises a pharmaceutically acceptable carrier. In another embodiment, he pharmaceutical composition of further comprises: (a) at least two Aurora kinase inhibitors selected from the group consisting of alisertib, CYC116, tozasertib, ZM447439, GSK1070916, PF03814735, SNS314, AMG900, MLN8054, CCT129202, CCT137690, MK5108, PHA680632, danusertib, TAK901, barasertib, ENMD-2076, AT-9283, KW-2449, ilorasertib, chiauranib, LY3295668 (AK-01), and TT-00420; and (b) at least two therapeutic molecules selected from the group consisting of DCZ0415, an siRNA molecule complementary to at least a portion of an mRNA sequence encoded by the TRIP13 gene, a gRNA molecule complementary to at least a portion of the TRIP13 gene, and a chemotherapeutic agent.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 demonstrates the efficacy of Aurora kinase inhibitors in HPV-positive squamous carcinoma cell lines; FIG. 1A compares the differential efficacy of 439 drugs based on HPV status. Only 30 drugs (FIG. 1A; left column) demonstrated a significantly different effect (P≤0.05) in HPV-positive cell lines compared to HPV-negative cell lines. The number in each cell corresponds to the number of drugs in each category, and the color of each cell corresponds to the standardized Pearson residual measuring the strength of enrichment for the drugs allocated to each category. Orange indicates enrichment. Blue indicates depletion. The histogram on the left of FIG. 1A shows the hypergeometric log 10 P values; the red dashed line indicates P=0.05. FIG. 1B shows the hypergeometric log 10 P values for 439 drugs for the drugs divided into 39 categories. The dashed red line indicates P=0.05. FIG. 1C shows AOC_LD values for 2 Aurora kinase inhibitors, alisertib and barasertib. The AOC_LD values were used as continuous variables, and cell lines were grouped by HPV status. Box plots show the median AOC_LD levels and 95% confidence intervals across all cell lines in each category. Each data point represents 1 cell line.

FIG. 2 demonstrates that Aurora kinase inhibition induces cell death in HPV-positive, but not HPV-negative squamous carcinoma cell lines in vitro. FIG. 2A shows the results of Bromodeoxyuridine (BrDU)-TUNEL assays used to measure apoptosis with flow cytometry in 3 HPV-positive and 3 HPV-negative squamous carcinoma cell lines treated with 50 nmol/L barasertib or 200 nmol/L alisertib for 72 hours (FIG. 2A, left panel) or 2 doubling times (FIG. 2A, right panel). BrdU positive apoptotic cells were detected and quantified. The bar graph indicates the percentage of apoptotic cells with fragmented DNA in HPV-positive and HPV-negative cell lines. Values are the means±the standard deviations for 3 independent experiments. FIG. 2B and FIG. 2C show immunoblots of cleaved PARP (Cl. PARP) and cleaved caspase-3 (Cl. Caspase-3) levels in 3-HPV positive and 3 HPV-negative cell lines after barasertib or alisertib treatment for 2 doubling times (FIG. 2B) or 72 hours (FIG. 2C). FIG. 2D shows the results of the detection and quantification if senescent cells in 4 HPV-positive and 5 HPV-negative cell lines treated with 50 nmol/L barasertib or 200 nmol/L alisertib for 72 hours using flow cytometry. ** P≤0.005; **** P≤0.0001 (unpaired, 2-tailed Student t test). UM47, UMSCC47; GAPDH, glyceraldehyde 3-phosphate dehydrogenase.

FIG. 3 demonstrates that Aurora kinase A inhibition leads to tumor regression and apoptosis in vivo in HPV-positive PDX models, but not in an HPV-negative cell line. PDX tumors HOSC144 (FIG. 3A, FIG. 3E, FIG. 3F), HOSC19 (FIG. 3B), and HOSC156 (FIG. 3C) were implanted into the flanks of nude mice. After engraftment, mice were randomized and assigned into 1 of 2 treatment groups: vehicle (β-cyclodextrin, n=8) or alisertib (10 mg/kg, n=8). Mice were treated for 3 weeks by daily oral gavage. Tumor size was measured twice a week. FIG. 3A, FIG. 3B, and FIG. 3C show in vivo tumor growth curves for individual mice (left 3 panels). Survival (right panels) was measured using the Kaplan-Meier method. In FIG. 3D, HPV-negative UMSCC22A cells were injected subcutaneously (3×10⁶ cells) on the right flanks of the mice. Once the tumor volume reached ≥60 mm³, mice were randomized into treatment groups. Mice were treated with vehicle or 10 mg/kg alisertib by oral gavage for 5 days per week for 21 days. Tumor size was measured twice a week. FIG. 3E shows immunohistochemical staining for cleaved Ki67 (FIG. 3E, upper left panel) with quantification of proliferative cells by Ki-67 (FIG. 3E, bottom left panel). The Ki67(%) was calculated as the ratio of proliferative cells to total cells in each field, using five random fields. FIG. 3F shows immunohistochemical staining for cleaved caspase-3 (Cl. Caspase-3; FIG. 3F, upper right panel) with quantification of apoptotic cells by staining Cl caspase-3 (FIG. 3F, lower right panel). The apoptotic (%) was calculated as the ratio of apoptotic cells to total cells in each field, using five random fields in HOSC144 PDX tumors after 8 or 21 days of treatment with either vehicle or alisertib. ** P≤0.01; *** P≤0.001. Bars, 100 μM.

FIG. 4 shows the effect of Rb expression on Aurora kinase A inhibition-induced apoptosis. FIG. 4A shows the levels of pRb (5807/5811) protein expression in alisertib-sensitive and -resistant squamous cancer cell lines as defined by AOC_LD values. Each data point represents 1 cell line. Box plots show the median pRb (5807/5811) levels and 95% confidence intervals across all cell lines in the category. t-test P=0.0097. FIG. 4B, HPV-negative HNSCC cells (HN31, UMSCC4, and MDA886LN) were infected with distinct, lentivirus-based shRNAs (Sh2, Sh4, and Sh6) to stably knock down the expression of RB1 or were treated with control (Ctrl) shRNA. Cells were then treated with 300 nmol/L alisertib for 24 hours before annexin V-PE/7AAD staining and analysis with flow cytometry. The bar graph represents the ratio of apoptosis (late plus early) among different groups. Values are the means±the standard deviations for 3 independent experiments. *P<0.05; **P<0.005; unpaired, 2-tailed Student t-test. FIG. 4C, cells from FIG. 4B were treated with 300 nmol/L alisertib for 24 hours before lysis and immunoblotting with the indicated antibodies. FIG. 4D-E, HN31 cells were stably transfected with an E7 plasmid and treated with 300 nmol/L alisertib for 24 hours before lysis and immunoblotting with (FIG. 4D) the indicated antibodies or (FIG. 4E) annexin-PE staining to measure apoptosis. Values are the means±the standard deviations for 3 independent experiments. ** P≤0.01; *** P≤0.001. FIG. 4F, two HPV-positive cell lines were transfected with 10 nmol/L siRNA oligonucleotides specific to E7 or with control oligonucleotides. Twenty-four hours later, the cells were treated with 300 nmol/L alisertib for an additional 24 hours and then harvested and immunoblotted for the indicated proteins. Representative results from 3 independent experiments are shown. Cl, cleaved; Fl, full length; GAPDH, glyceraldehyde 3-phosphate dehydrogenase; Ox, overexpression.

FIG. 5 demonstrates that the alteration of mitotic checkpoint proteins rescues Rb-depleted cells from Aurora kinase inhibitor-induced apoptosis. FIG. 5A, two HPV-positive cell lines (CASKI and SIHA) were transfected with BUB1B or MAD2L1 siRNA and then treated for 24 hours with 300 nmol/L alisertib before lysis and immunoblotting with the indicated antibodies. FIG. 5B, TRIP13 was transiently overexpressed (Ox) in 2 HPV-positive cell lines that were treated for 24 hours with 300 nmol/L alisertib before lysis and immunoblotting for the indicated proteins. FIG. 5C, HPV-positive cell lines with TRIP13 overexpression were treated with 300 nmol/L alisertib for 32 hours and then stained for annexin V-PE/7AAD staining and analysis with flow cytometry to measure apoptosis. The bar graph represents the ratio of apoptosis (late plus early) among different groups. Values are the means±the standard deviations for 3 independent experiments. ** P≤0.01; *** P≤0.001; unpaired, 2-tailed Student t-test. FIG. 5D, HN31 (left panel) and MDA886LN (right panel) cells expressing RB1 or control shRNA were transfected with a plasmid for TRIP13. After 24 hours, cells were treated with 300 nmol/L alisertib and then lysed for immunoblotting. Cl, cleaved; Fl, full length; GAPDH, glyceraldehyde 3-phosphate dehydrogenase.

FIG. 6 demonstrates that disturbing the balance of TRIP13 and MAD2 enhances Aurora kinase inhibition-induced apoptosis. FIG. 6A, mRNA expressions of MAD2L1, BUBIB and TRIP13 in HNSCC HPV-positive (n=70) and HPV-negative (n=447) HNSCC patient tumors from the TCGA (left panel) and in HPV-positive (n=51) and HPV-negative OPSCC [(n=28), right panel]. FIG. 6B, FIG. 6C, TRIP13 was depleted by siRNA in HPV-positive and negative cell lines. Cells were then treated with 100 nmol/L alisertib for 36 hours before being subjected to lysis and immunoblotting with the indicated antibodies (FIG. 6B) or annexin-PE staining to measure apoptosis using flow cytometry (FIG. 6C). FIG. 6D, FIG. 6E, the MAD2L1 plasmid was transiently transfected into 2 HPV-positive cell lines that were then treated with 100 nmol/L alisertib for 24 hours before being subjected to lysis and immunoblotting for the indicated proteins (FIG. 6D) or annexin-PE staining to measure apoptosis (FIG. 6E). Values are the means±the standard deviations for 3 independent experiments (** P≤0.01). Cl, cleaved; Fl, full length; GAPDH, glyceraldehyde 3-phosphate dehydrogenase; Ox, overexpression. FIG. 6F, model of the synthetic lethality between TRIP13 and Aurora kinase A. HPV-positive, Rb deficient cancer cells rely on both TRIP13 and Aurora kinase activity to maintain mitotic fidelity, such that their combined inhibition will lead to a prolonged spindle assembly checkpoint (SAC), irreversible mitotic arrest, and cancer cell death.

FIG. 7 demonstrates that depletion of TRIP13 in combination with an Aurora kinase inhibitor in NSCLC cell lines expressing low levels of Rb protein experience significantly more apoptosis compared to NSCLC cell lines expressing high levels of Rb. FIG. 7A, TRIP13 was depleted by siRNA in NSCLC cell lines followed by treatment with 100 nmol/L alisertib for 36 hours prior to Annexin-PE staining to measure apoptosis and analysis with flow cytometry. FIG. 7B and FIG. 7C show the levels of protein expression from immunoblots in seven NSCLC cell lines quantitated and normalized for β-actin and then positive control. Protein levels were compared to drug sensitivity combination.

FIG. 8 demonstrates that inhibition of TRIP13 using a small molecule inhibitor in combination with Aurora kinase inhibition in HPV-positive squamous cancers provides a synergistic effect. The combined effect of DCZ0415 and alisertib was examined in two HPV− negative (FADU and MDA886LN) and three HPV-positive (ME180, Caski, UM47) cell lines. Cells were treated with 100 nmol/L alisertib and/or 20 μmol/L DCZ0415 before annexin V-PE/7AAD staining and analysis with flow cytometry. The bar graph represents the ratio of apoptosis (late plus early) among different groups. Values are the means±the standard deviations for 3 independent experiments. *P<0.05; **P<0.005; unpaired, 2-tailed Student t-test.

### BRIEF DESCRIPTION OF THE SEQUENCES

SEQ ID NO:1 is a representative antisense sequence that may be used to reduce the expression of an Rb1 protein.

SEQ ID NO:2 is a representative antisense sequence that may be used to reduce the expression of an Rb1 protein.

SEQ ID NO:3 is a representative antisense sequence that may be used to reduce the expression of an Rb1 protein.

### DETAILED DESCRIPTION OF THE INVENTION

The present disclosure provides methods and compositions for the treatment of HPV-driven cancers. HPV-driven cancers, which are Rb-deficient due to the expression of the viral protein E7, depend upon the mitotic checkpoint complex for survival following Aurora kinase inhibition. This vulnerability results in a selective sensitivity to the combination of Aurora kinase inhibition and depletion of the MAD2L1 regulator TRIP13. This combination surprisingly results in substantial cancer cell death in HPV-positive, but not HPV-negative cancer cells and may thus improve rates of durable clinical responses. By sparing normal cells that express Rb and have unaltered levels of MAD2L1, this combination may reduce treatment toxicity among patients with HPV-positive cancer. The findings are also to the treatment of patients with other cancers with Rb pathway defects, including non-small cell lung cancer. The entire disclosure of Ghosh, et al., Clinical Cancer Research 28:4479-93, 2022, including all Supplementary Materials, is specifically incorporated by reference in its entirety.

### A. Therapeutic Compounds or Compositions and Administration Thereof

In accordance with the present disclosure, any Aurora kinase inhibitor may be used to treat or prevent an HPV-driven cancer. Non-limiting examples of Aurora kinase inhibitors include alisertib, CYC116, tozasertib, ZM447439, GSK1070916, PF03814735, SNS314, AMG900, MLN8054, CCT129202, CCT137690, MK5108, PHA680632, danusertib, TAK901, barasertib, ENMD-2076, AT-9283, KW-2449, ilorasertib, chiauranib, LY3295668 (AK-01), and TT-00420. According to the present disclosure, any therapeutic molecule that may be used to treat or prevent an HPV-driven cancer by decreasing Aurora kinase or TRIP13 protein expression or inhibiting its function. Non-limiting examples of such therapeutic molecules include a protein, a peptide, a polypeptide, an RNA molecule, a peptidomimetic, an siRNA molecule, a gRNA molecule, or a small molecule, and the like. In some embodiments, such therapeutic compounds target Aurora kinase or TRIP13 mRNA or TRIP13 or Aurora kinase protein to reduce the activity of TRIP13 or Aurora kinase in a cell or individual subject. In one embodiment, the inhibitor of TRIP13 function is the small molecule inhibitor DCZ0415. In another embodiment, liposomes or viral vectors may be used to provide therapeutic molecules of interest to a subject for targeting of TRIP13 or Aurora kinase. In further embodiments, a therapeutic compound described herein may be useful for targeting cells in the subject. In some embodiments, an siRNA for use according to the present disclosure may be complementary to a portion of the mRNA sequence encoded by the TRIP13 or Aurora kinase gene. In certain embodiments, a gRNA for use according to the present disclosure may be complementary to at least a portion the TRIP13 or Aurora kinase gene. In an embodiment, the siRNA molecule or gRNA molecule functions to inhibit TRIP13 or Aurora kinase in a subject.

The term “inhibitor” as used herein refers to an inhibitory molecule identified using an in vitro or in vivo assay. Inhibitors are compounds that may bind to, partially or totally block activity, decrease, prevent, delay activation, inactivate, desensitize, or down regulate the activity or expression of Aurora kinase or TRIP13. In some embodiments, assays for inhibitors may include expressing HPV-related proteins, Aurora kinase, or TRIP13 in vitro, applying putative inhibitor compounds, and then determining the functional effects on activity, as described herein. Test samples or assays that are treated with a potential inhibitor may be compared to a control sample lacking the inhibitor in order to determine the extent of inhibition. Control samples to which a test sample or assay is compared may be assigned a relative activity value of 100%. Inhibition is achieved, in certain embodiments, when the activity value of the test sample relative to the control sample is less than about 90%, including about 85%, about 80% about 75%, about 70%, about 65%, about 60%, about 55%, about 50%, about 45%, about 40%, about 35%, about 30%, about 25%, about 20%, about 15%, about 10%, about 5%, and about 0%.

Any method of interfering with Aurora kinase or TRIP13 activity in a subject, known in the art may be useful in accordance with the compositions and methods provided by the present disclosure. In other embodiments, a therapeutic molecule may be combined with a non-naturally occurring pharmaceutically acceptable carrier such as one described herein. In some embodiments, treatment methods of the present invention involve direct delivery of such a therapeutic molecule or compound. In other embodiments, treatment methods of the present invention may involve direct delivery of, for example, a vector expressing a functional copy of a therapeutic compound or molecule, such as an siRNA or gRNA molecule, for example a vector expressing an siRNA or gRNA molecule to reduce or eliminate the function of Aurora kinase or TRIP13. In further embodiments, treatment may comprise any combination of delivery of a therapeutic compound as described herein, delivery of a vector expressing a therapeutic compound, or a combination of these with any known treatment for HPV-positive cancers, including but not limited to administering an immunotherapy, a targeted cancer therapy, a chemotherapy, a radiation therapy, and surgery.

Therapeutic compounds or compositions within the scope of the present disclosure may also contain other compounds, which may be biologically active or inactive. For example, one or more small molecule inhibitors, siRNAs, or gRNAs described herein may be present, within a composition according to the disclosure. Therapeutic compounds or compositions may generally be used for prophylactic and/or therapeutic purposes. Embodiments of the disclosure provide therapeutic compounds or compositions for treatment of an HPV-driven pathological state. In certain embodiments, therapeutic compounds or compositions provided by the disclosure are useful in methods of treating or preventing an HPV-driven cancer, non-limiting examples of which include head and neck squamous cell carcinoma, cervical squamous cell carcinoma, anal squamous cell carcinoma, penile squamous cell carcinoma, and vulvar squamous cell carcinoma.

Therapeutic compounds or compositions may be provided to a subject in a single dose or multiple doses and as such provided in single-dose or multi-dose containers, such as sealed ampules or vials. Such containers may be sealed to preserve sterility of the composition until use. In general, compositions as described herein may be stored as suspensions, solutions, or emulsions in oily or aqueous vehicles. Alternatively, such a composition may be stored in a freeze-dried condition requiring only the addition of a sterile liquid carrier immediately prior to use.

As described herein, a therapeutic composition may be combined with a pharmaceutically acceptable carrier. In some embodiments, a pharmaceutically acceptable carrier as described herein is non-naturally occurring. The selection of a suitable carrier may be determined in part by the particular composition being administered (e.g., small molecule inhibitors, protein, modulatory compounds, siRNA molecules, gRNA molecules, or the like), as well as by the particular method used to administer the composition. Accordingly, a wide variety of suitable formulations of therapeutic compositions are available that may of use in the present disclosure. Administration may be in any convenient manner, e.g., by injection, oral administration, inhalation, transdermal application, or rectal administration.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended subject, and aqueous and nonaqueous sterile suspensions that can include suspending agents, solubilizers, thickening agents, stabilizers, and preservatives. In the practice of this disclosure, compositions may be administered, for example, by intravenous infusion into the circulation, orally, topically, or intraperitoneally.

Such compositions may also comprise buffers (e.g., neutral buffered saline or phosphate buffered saline), carbohydrates (e.g., glucose, mannose, sucrose or dextrans), mannitol, proteins, polypeptides or amino acids such as glycine, antioxidants, bacteriostats, chelating agents such as EDTA or glutathione, adjuvants (e.g., aluminum hydroxide), solutes that render the formulation isotonic, hypotonic, or weakly hypertonic with the blood of a subject, suspending agents, thickening agents, and/or preservatives. Alternatively, compositions of the present disclosure may be formulated as a lyophilizate. Compounds may also be encapsulated within liposomes using methods known in the art.

Injection solutions and suspensions may be prepared from sterile powders, granules, and tablets as described herein. An injection as described herein may involve a suspension of one or more of a purified or non-purified solution of a protein, nucleic acid, or other type of molecule as described herein. An injection solution may also contain a pharmaceutically acceptable carrier as described herein.

Formulations suitable for oral administration may consist of (a) liquid solutions, such as an effective amount of a small molecule inhibitor or other type of molecule suspended in diluents, such as water, saline or PEG 400; (b) capsules or tablets, each containing a predetermined amount of the active ingredient, as liquids, solids, granules, or gelatin; (c) suspensions in an appropriate liquid; or (d) suitable emulsions. Tablet forms may include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms may comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

The compound of choice, alone or in combination with other suitable components, may be made into aerosol formulations to be administered via inhalation. Aerosol formulations may be placed into pressurized acceptable propellants, such as dichlorodifluoromethane, propane, nitrogen, and the like.

The dose administered to a subject in the context of the present disclosure should be sufficient to affect a beneficial therapeutic response in the subject over time. The dose will be determined by the efficacy of the particular molecule employed and the condition of the subject, as well as the body weight and/or surface area of the patient to be treated. The size of the dose also may be determined by the existence, nature, and extent of any adverse side-effects that accompany the administration of a particular molecule or therapeutic compound in a particular subject.

For administration, compounds of the present disclosure can be administered at a rate determined by the LD-50 of the molecule or therapeutic compound, and the side-effects thereof at various concentrations, as applied to the mass and overall health of the subject. Administration may be accomplished via single, multiple, or divided doses.

As used herein, a “therapeutic compound” or “therapeutic composition” refers to a molecule, such as a small molecule inhibitor, an RNA molecule, protein, a peptide, a polypeptide, an siRNA molecule, or a gRNA molecule that inhibits the expression or function of Aurora kinase or TRIP13. Such a compound or composition is meant to encompass a composition suitable for administration to a subject, such as a mammal, or particularly a human subject. In general, a therapeutic composition is sterile, and preferably free of contaminants that are capable of eliciting an undesirable response within the subject (e.g., the compound(s) in the immunogenic composition is pharmaceutical grade). Therapeutic compositions may be designed for administration to subjects in need thereof via a number of different routes of administration including oral, intravenous, buccal, rectal, parenteral, intraperitoneal, intradermal, intratracheal, intramuscular, subcutaneous, inhalational, and the like.

As used herein, “subject” or “patient” refers to animals, including humans, who are treated with the therapeutic compounds or compositions or in accordance with the methods described herein. For diagnostic or research applications, a wide variety of mammals may be suitable subjects, including rodents (e.g., mice, rats, hamsters), rabbits, primates, and swine, such as inbred pigs and the like.

As used herein, a “biological sample” or “sample” may include blood and blood parts including, but not limited to serum, plasma, platelets, or red blood cells; sputum, mucosa, tissue, cultured cells, including primary cultures, and transformed cells; biological fluids, stool, and urine. A biological sample may also include sections of tissues such as biopsy and autopsy samples, and frozen sections taken for histologic purposes. A biological sample may be obtained from a eukaryotic organism, such as a human. Any tissue appropriate for use in accordance with the disclosure may be used, for instance, tumor tissue, skin, brain, spinal cord, adrenals, pectoral muscle, lung, heart, liver, duodenum, small intestine, large intestine, kidney, spleen, pancreas, adrenal gland, bone marrow, lumbosacral spinal cord, or blood.

As used herein, a “pharmaceutically acceptable carrier,” “pharmaceutically acceptable adjuvant,” or “adjuvant” refers to refers to reagents, cells, compounds, materials, compositions, and/or dosage forms that are not only compatible with the agents to be administered therapeutically, but also are, within the scope of sound medical judgment, suitable for use in contact with the tissues of human beings and animals without excessive toxicity, irritation, allergic response, or other complication commensurate with a reasonable benefit/risk ratio. Also included may be an agent that modifies the effect of other agents and is useful in preparing a therapeutic compound or composition that is generally safe, non-toxic, and neither biologically nor otherwise undesirable. Such an agent may be added to a therapeutic composition to modify the immune response of a subject by boosting the response and providing longer-lasting protection. Such an agent may include an excipient, diluent, carrier, or adjuvant that is acceptable for pharmaceutical use. Such an agent may be non-naturally occurring, or may be naturally occurring, but not naturally found in combination with other agents in the immunogenic composition.

The term “isolated compound” means a compound which has been substantially separated from, or enriched relative to, other compounds with which it occurs in nature. Isolated compounds are usually at least about 80%, at least 90% pure, at least 98% pure, or at least about 99% pure, by weight.

The term “unit dosage form,” as used herein, refers to physically discrete units suitable as unitary dosages for animal subjects, each unit containing a predetermined quantity of a compound calculated in an amount sufficient to produce the desired effect in association with a pharmaceutically acceptable diluent, carrier, or vehicle. The specifications for unit dosage forms depend on the particular compound employed, the route and frequency of administration, the effect to be achieved, and the pharmacodynamics associated with each compound in the host.

As used herein, “cell culture,” “in culture,” or “cultured” refers generally to cells taken from a living organism and grown under controlled conditions. A “primary cell culture” is a culture of cells, tissues, or organs taken directly from an organism before the first subculture. Cells are expanded in culture when they are placed in a growth medium under conditions that facilitate growth and/or division, resulting in a larger population of the cells. When cells are expanded in culture, the rate of cell proliferation is sometimes measured by the amount of time needed for the cells to double in number, referred to as “doubling time.”

As used herein, “introducing,” “delivering,” and “administering” refer to the therapeutic introduction of a therapeutic compound or composition as described herein to a subject. Administration may take place by any route that provides the therapeutic compound to the circulation of the subject in accordance with the invention.

The phrase “effective amount” refers to a concentration or amount of a therapeutic compound or composition as described herein, reagent, or other agent, that is effective for producing an intended result, including treatment of HPV-driven cancers as described herein. With respect to the administration of a therapeutic compound as disclosed herein, an effective amount may be any effective range or concentration. The exact dose will depend on the purpose of the treatment, and one of skill in the art will be able to determine such a dose using techniques known in the art.

As used herein, “expression” refers to the combination of intracellular processes, including transcription and translation undergone by a coding DNA molecule such as a structural gene to produce a polypeptide or functional nucleic acid (e.g., an RNAi, gRNA, antisense molecule, ribozyme, aptamer, etc.).

### B. Gene Suppression

As used herein the phrase “gene suppression,” is intended to refer to any of the well-known methods for reducing the levels of protein produced as a result of gene transcription to mRNA and subsequent translation of the mRNA. Gene suppression is also intended to mean the reduction of protein expression from a gene or a coding sequence including posttranscriptional gene suppression and transcriptional suppression. Post-transcriptional gene suppression is mediated by the homology between of all or a part of a mRNA transcribed from a gene or coding sequence targeted for suppression and the corresponding double stranded RNA used for suppression and refers to the substantial and measurable reduction of the amount of available mRNA available in the cell for binding by ribosomes. The transcribed RNA can be in the sense orientation to effect what is called co-suppression, in the anti-sense orientation to effect what is called anti-sense suppression, or in both orientations producing a double stranded RNA (dsRNA) to effect what is called RNA interference (RNAi). Transcriptional suppression is mediated by the presence in the cell of a dsRNA, a gene suppression agent, exhibiting substantial sequence identity to a promoter DNA sequence or the complement thereof to effect what is referred to as promoter trans suppression. Gene suppression may be effective against a native gene associated with a trait, e.g., to suppress expression of a TRIP13 or Aurora kinase protein. Sequences of the TRIP13 and Aurora kinase genes and sequences encoded by the TRIP13 and Aurora kinase genes are known in the art and any such gene, mRNA, or protein sequence may be targeted according to the present disclosure. One of skill the art would understand that due to, for example, certain genetic polymorphisms or mutations that may be present in the human population, the gene, encoded mRNA molecule, or encoded polypeptide sequence may comprise a sequence having at least about 70%, about 75%, about 80%, about 85%, about 90%, about 91%, about 92%, about 93%, about 94%, about 95%, about 96%, about 97%, about 98%, about 99%, or 100% sequence identity to a TRIP13 or Aurora kinase gene, a sequence encoded thereby, or a fragment thereof.

Post-transcriptional gene suppression may employ both sense-oriented and anti-sense-oriented, transcribed RNA, which is stabilized, e.g., as a hairpin and stem and loop structure. A preferred DNA construct for effecting post transcriptional gene suppression one in which a first segment encodes an RNA exhibiting an anti-sense orientation exhibiting substantial identity to a segment of a gene targeted for suppression, which is linked to a second segment encoding an RNA exhibiting substantial complementarity to the first segment. Such a construct would be expected to form a stem and loop structure by hybridization of the first segment with the second segment and a loop structure from the nucleotide sequences linking the two segments.

The present disclosure provides dsRNA or siRNA molecules for reduction or elimination of TRIP13 or Aurora kinase expression. siRNA technology is known in the art and used to study inhibition of, for example, apolipoprotein receptors such as ApoCIII (Gaudet et al., N Engl J Med 373(5):438-47, 2015; Graham et al., Circulation Res 112:1479-1490, 2013) ApoA (Tsimikas et al., Lancet 2015), or chemicals useful in accordance with the disclosure, such as PCSK9 (Miao et al., Arterioscler Thromb Vasc Biol 35(7):1589-96, 2015). The dsRNA or siRNA nucleotide sequences comprise double strands of polymerized ribonucleotide and may include modifications to either the phosphate-sugar backbone or the nucleoside. Modifications in RNA structure may be tailored to allow specific genetic inhibition. In one embodiment, the dsRNA molecules may be modified through an enzymatic process so the siRNA molecules may be generated. The siRNA can efficiently mediate the down-regulation effect for some target genes. This enzymatic process may be accomplished by utilizing an RNAse III enzyme or a DICER enzyme, present in the cells of an individual in the eukaryotic RNAi pathway. Both the DICER enzyme and RNAse III, naturally occurring in an individual or made through recombinant DNA techniques, cleave larger dsRNA strands into smaller oligonucleotides. The DICER enzymes specifically cut the dsRNA molecules into siRNA pieces each of which is about 19-25 nucleotides in length while the RNAse III enzymes normally cleave the dsRNA molecules into 12-15 base-pair siRNA. The siRNA molecules produced by the either of the enzymes have 2 to 3 nucleotide 3′ overhangs, and 5′ phosphate and 3′ hydroxyl termini. The siRNA molecules generated by RNAse III enzyme are the same as those produced by Dicer enzymes in the eukaryotic RNAi pathway and are hence then targeted and degraded by an inherent cellular RNA-degrading mechanism after they are subsequently unwound, separated into single-stranded RNA, and hybridized with the RNA sequences transcribed by the target gene. This process results in the effective degradation or removal of the RNA sequence encoded by the nucleotide sequence of the target gene. The outcome is the silencing of a particularly targeted nucleotide sequence within the individual.

Inhibition of a target gene using the stabilized dsRNA technology of the present disclosure is sequence-specific in that nucleotide sequences corresponding to the duplex region of the RNA are targeted for genetic inhibition. RNA containing a nucleotide sequence identical to a portion of the target gene is preferred for inhibition. RNA sequences with insertions, deletions, and single point mutations relative to the target sequence have also been found to be effective for inhibition. In performance of the present disclosure, it is preferred that the inhibitory dsRNA and the portion of the target gene share at least from about 80% sequence identity, about 85% identity, about 90% sequence identity, about 91% identity, about 92% identity, about 93% identity, about 94% identity, or from about 95% sequence identity, about 96% identity, about 97% identity, about 98% identity, about 99% sequence identity, or about 100% sequence identity. Alternatively, the duplex region of the RNA may be defined functionally as a nucleotide sequence that is capable of hybridizing with a portion of the target gene transcript. A less than full length sequence exhibiting a greater homology compensates for a longer less homologous sequence. The length of the identical nucleotide sequences may be at least about 15, 16, 17, 18, 19, 20, 21, 22, 23, 24, 25, 30, 35, 40, 45, 50, 60, 70, 80, 90, 100, 200, 300, 400, 500, or at least about 1000 bases. Normally, a sequence of greater than 15-100 nucleotides may be used, or a sequence of greater than about 200-300 nucleotides, or a sequence of greater than about 500-1000 nucleotides, depending on the size of the target gene. A dsRNA or siRNA as described herein may be able to tolerate sequence variations that might be expected due to genetic mutation, polymorphism, or evolutionary divergence. The introduced nucleic acid molecule may not need to be absolute homology, may not need to be full length, relative to either the primary transcription product or fully processed mRNA of the target gene. Therefore, those skilled in the art will understand that 100% sequence identity between the RNA and the target gene is not required to practice the present disclosure.

The dsRNA or siRNA molecules may be synthesized either in vivo or in vitro. The dsRNA may be formed by a single self-complementary RNA strand or from two complementary RNA strands. Endogenous RNA polymerase of the cell may mediate transcription in vivo, or cloned RNA polymerase can be used for transcription in vivo or in vitro. Inhibition may be targeted by specific transcription in an organ, tissue, or cell type; stimulation of an environmental condition (e.g., infection, stress, temperature, chemical inducers); and/or engineering transcription at a developmental stage or age. The RNA strands may or may not be polyadenylated; the RNA strands may or may not be capable of being translated into a polypeptide by a cell's translational apparatus.

The RNA, dsRNA, siRNA, or miRNA of the present disclosure may be produced chemically or enzymatically by one skilled in the art through manual or automated reactions or in vivo in another organism. RNA may also be produced by partial or total organic synthesis; any modified ribonucleotide can be introduced by in vitro enzymatic or organic synthesis. The use and production of an expression construct are known in the art. If synthesized chemically or by in vitro enzymatic synthesis, the RNA may be purified prior to introduction into the cell. For example, RNA can be purified from a mixture by extraction with a solvent or resin, precipitation, electrophoresis, chromatography, or a combination thereof. Alternatively, the RNA may be used with no or a minimum of purification to avoid losses due to sample processing. The RNA may be dried for storage or dissolved in an aqueous solution. The solution may contain buffers or salts to promote annealing, and/or stabilization of the duplex strands.

As used herein, the term “nucleic acid” refers to a single or double-stranded polymer of deoxyribonucleotide or ribonucleotide bases read from the 5′ to the 3′ end. The “nucleic acid” may also optionally contain non-naturally occurring or altered nucleotide bases that permit correct read through by a polymerase and do not reduce expression of a polypeptide encoded by that nucleic acid. The term “nucleotide sequence” or “nucleic acid sequence” refers to both the sense and antisense strands of a nucleic acid as either individual single strands or in the duplex. The term “ribonucleic acid” (RNA) is inclusive of RNAi (inhibitory RNA), dsRNA (double stranded RNA), siRNA (small interfering RNA), mRNA (messenger RNA), miRNA (micro-RNA), tRNA (transfer RNA, whether charged or discharged with a corresponding acylated amino acid), and cRNA (complementary RNA) and the term “deoxyribonucleic acid” (DNA) is inclusive of cDNA and genomic DNA and DNA-RNA hybrids. The words “nucleic acid segment”, “nucleotide sequence segment”, or more generally “segment” will be understood by those in the art as a functional term that includes both genomic sequences, ribosomal RNA sequences, transfer RNA sequences, messenger RNA sequences, operon sequences and smaller engineered nucleotide sequences that express or may be adapted to express, proteins, polypeptides, or peptides.

### C. Genome Editing

Any site or locus within the genome of an HPV-positive cancer cell may potentially be chosen for making a genomic edit (or gene edit) or site-directed integration of a transgene, construct, or transcribable DNA sequence. As used herein, a “target site” for genome editing or site-directed integration refers to the location of a polynucleotide sequence within a cancer cell genome that is bound and cleaved by a site-specific nuclease to introduce a double-stranded break (DSB) or single-stranded nick into the nucleic acid backbone of the polynucleotide sequence and/or its complementary DNA strand within the genome. A target site may comprise, for example, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 29, or at least 30 consecutive nucleotides. A “target site” for an RNA-guided nuclease may comprise the sequence of either complementary strand of a double-stranded nucleic acid (DNA) molecule or chromosome at the target site. A site-specific nuclease may bind to a target site, such as via a non-coding guide RNA (e.g., without being limiting, a CRISPR RNA (crRNA) or a single-guide RNA (sgRNA) as described further herein). A non-coding guide RNA provided herein may be complementary to a target site (e.g., complementary to either strand of a double-stranded nucleic acid molecule or chromosome at the target site). It will be appreciated that perfect identity or complementarity may not be required for a non-coding guide RNA to bind or hybridize to a target site. For example, at least 1, at least 2, at least 3, at least 4, at least 5, at least 6, at least 7, or at least 8 mismatches (or more) between a target site and a non-coding RNA may be tolerated. A “target site” also refers to the location of a polynucleotide sequence within a genome that is bound and cleaved by any other site-specific nuclease that may not be guided by a non-coding RNA molecule, such as a zinc finger nuclease (ZFN), a transcription activator-like effector nuclease (TALEN), a meganuclease, etc., to introduce a DSB or single-stranded nick into the polynucleotide sequence and/or its complementary DNA strand. As used herein, a “target region” or a “targeted region” refers to a polynucleotide sequence or region that is flanked by two or more target sites. Without being limiting, in some embodiments a target region may be subjected to a mutation, deletion, insertion, substitution, inversion, or duplication. As used herein, “flanked” when used to describe a target region of a polynucleotide sequence or molecule, refers to two or more target sites of the polynucleotide sequence or molecule surrounding the target region, with one target site on each side of the target region.

As used herein, a “targeted genome editing technique” refers to any method, protocol, or technique that allows the precise and/or targeted editing of a specific location in a genome of an cancer cell (i.e., the editing is largely or completely non-random) using a site-specific nuclease, such as a meganuclease, a zinc-finger nuclease (ZFN), an RNA-guided endonuclease (e.g., the CRISPR/Cas9 or Cas12a system), a TALE (transcription activator-like effector)-endonuclease (TALEN), a recombinase, or a transposase. In particular embodiments, a “targeted genome editing technique” refers to an RNA-guided Cas12a system. As used herein, “editing” or “genome editing” refers to generating a targeted mutation, deletion, insertion, substitution, inversion or duplication of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 75, at least 100, at least 250, at least 500, at least 1000, at least 2500, at least 5000, at least 10,000, or at least 25,000 nucleotides of an endogenous cancer cell genome nucleic acid sequence. As used herein, “editing” or “genome editing” may also encompass the targeted insertion or site-directed integration of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 75, at least 100, at least 250, at least 500, at least 750, at least 1000, at least 1500, at least 2000, at least 2500, at least 3000, at least 4000, at least 5000, at least 10,000, or at least 25,000 nucleotides into the endogenous genome of a cancer cell. An “edit” or “genomic edit” in the singular refers to one such targeted mutation, deletion, insertion, substitution, inversion, or duplication, whereas “edits” or “genomic edits” refers to two or more targeted mutation(s), deletion(s), insertion(s), substitution(s), inversion(s), and/or duplication(s), with each “edit” being introduced via a targeted genome editing technique.

According to some embodiments, a site-specific nuclease may be co-delivered with a donor template molecule to serve as a template for making a desired edit, mutation, or insertion into the genome at the desired target site through repair of the double strand break (DSB) or nick created by the site-specific nuclease. According to some embodiments, a site-specific nuclease may be co-delivered with a DNA molecule comprising a selectable or screenable marker gene.

A site-specific nuclease may be an RNA-guided nuclease. According to some embodiments, an RNA-guided endonuclease may be selected from the group consisting of Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, CasX, CasY, and homologs or modified versions of any thereof, as well as Argonaute proteins (non-limiting examples of Argonaute proteins include Thermus thermophilus Argonaute (TtAgo), Pyrococcus furiosus Argonaute (PfAgo), Natronobacterium gregoryi Argonaute (NgAgo), and homologs or modified versions of any thereof). According to some embodiments, an RNA-guided endonuclease is a Cas9 or Cpf1 (also referred to herein as Cas12a) enzyme. The RNA-guided nuclease may be delivered as a protein or a recombinant DNA construct comprising a polynucleotide sequence encoding said nuclease, with or without a guide RNA; or the guide RNA may be complexed with the RNA-guided nuclease enzyme and delivered as a ribonucleoprotein (RNP).

For RNA-guided endonucleases, a guide RNA molecule may be further provided to direct the endonuclease to a target site in the genome of the cancer cell via base-pairing or hybridization to cause a DSB or nick at or near the target site. As described herein, the guide RNA may be transformed or introduced into a cancer cell or tissue as a gRNA molecule, or as a recombinant DNA molecule, construct or vector comprising a transcribable DNA sequence encoding one or more guide RNAs operably linked to a single promoter or individual promoters. As understood in the art, a guide RNA may comprise, for example, a CRISPR RNA (crRNA), a single-chain guide RNA (sgRNA), or any other RNA molecule that may guide or direct an endonuclease to a specific target site in the genome. A prototypical CRISPR associated protein, Cas9 from S. pyogenes, naturally binds two RNAs, a CRISPR RNA (crRNA) guide and a trans-acting CRISPR RNA (tracrRNA), to assemble a CRISPR ribonucleoprotein (crRNP). In comparison, the CRISPR-Cas12a system does not require a trans-activating crispr RNA (tracrRNA) for biogenesis of mature crRNA. Instead, the RuvC endonuclease domain of Cas12a processes its mature crRNA directly. A “single-chain guide RNA” (or “sgRNA”) is an RNA molecule comprising a crRNA covalently linked a tracrRNA by a linker sequence, which may be expressed as a single RNA transcript or molecule. The guide RNA comprises a guide or targeting sequence (also referred to herein as a “spacer sequence”) that is identical or complementary to a target site within the cancer cell genome, such as at or near a gene. The guide RNA is typically a non-coding RNA molecule that does not encode a protein. The guide sequence of the guide RNA may be at least 10 nucleotides in length, such as 12-40 nucleotides, 12-30 nucleotides, 12-20 nucleotides, 12-35 nucleotides, 12-30 nucleotides, 15-30 nucleotides, 17-30 nucleotides, or 17-25 nucleotides in length, or about 12, 13, 14, 15, 16, 17, 18, 19, 20, 21, 22, 23, 24, 25 or more nucleotides in length. The guide sequence may be at least 95%, at least 96%, at least 97%, at least 99% or 100% identical or complementary to at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides of a DNA sequence at the genomic target site.

As mentioned above, a target gene for genome editing may be any gene of interest present in a cancer cell. For knockdown mutations of the gene of interest through genome editing, an RNA-guided endonuclease may be targeted to an upstream or downstream sequence, such as a promoter and/or enhancer sequence, or an intron, 5′UTR, and/or 3′UTR sequence of the gene to mutate one or more promoter and/or regulatory sequences of the gene to affect or reduce its level of expression. Similarly, mutations of the gene of interest through genome editing, an RNA-guided endonuclease may be targeted to a transcribable DNA sequence (i.e., a transcribable region) of said gene, such as a region of the gene comprising a coding sequence, a specific DNA sequence encoding a protein domain, an exon region, an intron region, or a combination thereof. For example, in certain embodiments a transcribable DNA sequence targeted for genome editing may comprise an exon/intron boundary or may be in close proximity to an exon/intron boundary. If the resulting modification spans an exon/intron boundary, the modification may be referred to as a modification in an exon region and an intron region. For genetic modification of the gene of interest, a guide RNA may be used, which comprises a guide sequence that is at least 90%, at least 95%, at least 96%, at least 97%, at least 99% or 100% identical or complementary to at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides of the sequence of the TRIP13 or Aurora kinase gene or a sequence complementary thereto, although alternative splicing and different exon/intron boundaries may occur. As used herein, the term “consecutive” in reference to a polynucleotide or protein sequence means without deletions or gaps in the sequence.

As used herein, with respective to a given sequence, a “complement”, a “complementary sequence” and a “reverse complement” are used interchangeably. All three terms refer to the inversely complementary sequence of a nucleotide sequence, i.e., to a sequence complementary to a given sequence in reverse order of the nucleotides.

Antisense RNA molecules are single-stranded nucleic acids which can combine with a sense RNA strand or sequence or mRNA to form duplexes due to complementarity of the sequences. The term “antisense strand” refers to a nucleic acid strand that is complementary to the “sense” strand. The “sense strand” of a gene or locus is the strand of DNA or RNA that has the same sequence as an RNA molecule transcribed from the gene or locus (with the exception of uracil in RNA and thymine in DNA).

A protospacer-adjacent motif (PAM) may be present in the genome immediately adjacent and upstream to the 5′ end of the genomic target site sequence complementary to the targeting sequence of the guide RNA—i.e., immediately downstream (3′) to the sense (+) strand of the genomic target site (relative to the targeting sequence of the guide RNA) as known in the art. See, e.g., Wu et al. (Quant Biol. 2(2):59-70, 2014). The genomic PAM sequence on the sense (+) strand adjacent to the target site (relative to the targeting sequence of the guide RNA) may comprise 5′-NGG-3′ for Cas9; or 5′-TTTN-3′ for Cas12a. However, the corresponding sequence of the guide RNA (i.e., immediately downstream (3′) to the targeting sequence of the guide RNA) may generally not be complementary to the genomic PAM sequence.

As used herein, a “donor molecule”, “donor template”, or “donor template molecule” (collectively a “donor template”), which may be a recombinant polynucleotide, DNA or RNA donor template or sequence, is defined as a nucleic acid molecule having a homologous nucleic acid template or sequence (e.g., homology sequence) and/or an insertion sequence for site-directed, targeted insertion or recombination into the genome of a cancer cell via repair of a nick or DSB in the genome of a cancer cell. A donor template may be a separate DNA molecule comprising one or more homologous sequence(s) and/or an insertion sequence for targeted integration, or a donor template may be a sequence portion (i.e., a donor template region) of a DNA molecule further comprising one or more other expression cassettes, genes/transgenes, and/or transcribable DNA sequences. For example, a “donor template” may be used for site-directed integration of a transgene or construct, or as a template to introduce a mutation, such as an insertion, deletion, substitution, etc., into a target site within the genome of a cancer cell. A targeted genome editing technique provided herein may comprise the use of one or more, two or more, three or more, four or more, or five or more donor molecules or templates. A donor template provided herein may comprise at least one, at least two, at least three, at least four, at least five, at least six, at least seven, at least eight, at least nine, or at least ten gene(s) or transgene(s) and/or transcribable DNA sequence(s). Alternatively, a donor template may comprise no genes, transgenes, or transcribable DNA sequences.

Any method known in the art for site-directed integration may be used with the present disclosure. In the presence of a donor template molecule with an insertion sequence, the DSB or nick can be repaired by homologous recombination between homology arm(s) of the donor template and the genome, or by non-homologous end joining (NHEJ), resulting in site-directed integration of the insertion sequence into the genome to create the targeted insertion event at the site of the DSB or nick. Thus, site-specific insertion or integration of a transgene, transcribable DNA sequence, construct, or sequence may be achieved if the transgene, transcribable DNA sequence, construct or sequence is located in the insertion sequence of the donor template.

The introduction of a DSB or nick may also be used to introduce targeted mutations in the genome of a cancer cell. According to this approach, mutations, such as deletions, insertions, substitutions, inversions, and/or duplications may be introduced at a target site via imperfect repair of the DSB or nick to produce a genetic modification within a gene. Such mutations may be generated by imperfect repair of the targeted locus even without the use of a donor template molecule. A modification of a gene may be achieved by inducing a DSB or nick at or near the endogenous locus of the gene that results in expression of a non-functional protein, interfering protein, or a protein having reduced, disrupted, or altered activity as compared to a protein expressed from the gene lacking said modification.

Similarly, such targeted mutations of a gene may be generated with a donor template molecule to direct a particular or desired mutation at or near the target site via repair of the DSB or nick. The donor template molecule may comprise a homologous sequence with or without an insertion sequence and comprising one or more mutations, such as one or more deletions, insertions, substitutions, inversions, and/or duplications, relative to the targeted genomic sequence at or near the site of the DSB or nick. For example, targeted mutations of a gene may be achieved by deleting, inserting, substituting, inverting, or duplicating at least a portion of the gene, such as by introducing a frame shift or premature stop codon into the coding sequence of the gene or introducing a modification into a transcribable DNA sequence. A deletion of a portion of a gene may also be introduced by generating DSBs or nicks at two target sites and causing a deletion of the intervening target region flanked by the target sites. A modification of a targeted gene may result in expression of a non-functional protein, interfering protein, or a protein having reduced, disrupted, or altered activity as compared to a protein expressed from the gene lacking said modification.

The term “about” is used to indicate that a value includes the standard deviation of the mean for the device or method being employed to determine the value. The use of the term “or” in the claims is used to mean “and/or” unless explicitly indicated to refer to alternatives only or the alternatives are mutually exclusive. When used in conjunction with the word “comprising” or other open language in the claims, the words “a” and “an” denote “one or more,” unless specifically noted otherwise. The terms “comprise,” “have,” and “include” are open-ended linking verbs. Any forms or tenses of one or more of these verbs, such as “comprises,” “comprising,” “has,” “having,” “includes,” and “including,” are also open-ended. For example, any method that “comprises,” “has,” or “includes” one or more steps is not limited to possessing only those one or more steps and also covers other unlisted steps. Similarly, any system or method that “comprises,” “has,” or “includes” one or more components is not limited to possessing only those components and covers other unlisted components.

Other objects, features, and advantages of the present disclosure are apparent from detailed description provided herein. It should be understood, however, that the detailed description and any specific examples provided, while indicating specific embodiments of the disclosure, are given by way of illustration only, since various changes and modifications within the spirit and scope of the disclosure will become apparent to those skilled in the art from this detailed description. Any embodiment of the present disclosure may be used in combination with any other embodiment described herein.

All references herein are incorporated herein by reference in their entirety.

### EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Example 1

### Cells Lines and Reagents

HNSCC and cervical squamous carcinoma cell lines were obtained, maintained, and profiled as described previously (Zhao, et al., Clin Cancer Res 2011; 17(23):7248-64). Tests performed included RPPA (Mazumdar, et al., Mol Cancer Ther 2014; 13(11):2738-50; Kaiu, et al., Cancer Lett 2018; 431:64-72), RNA-seq (Kaiu, et al., Oncotarget 2017; 8(49):86369-83), HPV integration (Kau, etnal., Oncotarget 2017; 8(49):86369-83), and whole-exome sequencing (Zhang, et al., Cancer Lett 2017; 392:71-82). RPPA was performed on 28 cell lines in the HTDS in a single batch to avoid batch effects. No RPPA data were available for HN31, PCJ15B, UMSCCI, UMSCC22A, or UMSCC25. All cell lines were genotyped using short tandem repeat analysis, and all were mycoplasma-free at the time of testing as determined using a mycoplasma detection kit (Lonza, Walkersville, MD). All drugs, except those used in the HTDS, were purchased from Selleck Chemicals (Houston, TX), and prepared as 10 mmol/L stock solutions in DMS0. Antibodies are listed in Table 1.

TABLE 1
Antibodies used in the Examples.

| | Alternate | Gene | | Catalog |
|---|---|---|---|---|
| Official Ab name | Ab name | name | Supplier | # |
| Rb | Rb | RB1 | CST | 9309 |
| pRb (Ser807/811) | Rb_pS807_S811 | RB1 | CST | 9308 |
| p16INK4a (D7C1M) | p16INK4a | CDKN2A | CST | 80772 |
| Cyclin B | Cyclin-B1 | CCNB1 | CST | 4135 |
| p21 | p21 | CDKN1A | Santa Cruz | sc-397 |
| Cleaved PARP (Asp214) | Parp | Parp | CST | 94885 |
| PARP | PARP | PARP | CST | 9532 |
| Cleaved Caspase-3 | Caspase-3 | CASP3 | CST | 9661 |
| (Asp175) | | | | |
| BUB1B | BUB1B | BUB1B | CST | 4116 |
| MAD2L1 | MAD2 | MAD2 | CST | 4636 |
| TRIP13 | TRIP13 | TRIP13 | Santa Cruz | 514285 |
| Phospho-ULK1 (Ser555) | ULK1_pS757 | Ulk1 | CST | 5869 |
| Phospho-MEK1/2 | MEK1_pS217_{—} | MEK1 | CST | 3958 |
| (Ser217/221) | S221 | | | |
| c-Myc | c-Myc | MYC | Santa Cruz | sc-764 |
| Pan-MYC | MYC | MYC | abcam | ab195207 |
| PTEN | PTEN | PTEN | CST | 9552 |
| Tau | Tau | TAU | CST | 46687 |
| TSC1/Hamartin | TSC1 | TSC1 | CST | 4906 |
| PMS2 | PMS2 | PMS2 | Santa Cruz | sc-25315 |
| Beclin1 | Beclin | BECN1 | CST | 3738 |
| SMAD4 | SMAD4 | SMAD4 | CST | 46535 |
| Aurora A (D3E4Q) | Aurora A | Aurka | CST | 14475 |
| Aurora B/AIM1 | Aurora B | AURKB | CST | 3094 |
| HPV16 E7 | E7 | E7 | Gene Tex | GTX133411 |
| 3-Actin | 3-Actin | ACTB | CST | 4970 |
| GAPDH | GAPDH | GAPDH | CST | 5174 |

### Example 2

### Immunoblotting

Western blot analysis was performed as described previously (Ferrarotto, et al., Clin Cancer Res 2016; 22(7):1674-86). In brief, cells were lysed with ice-cold lysis buffer, and the lysates were centrifuged at 20,000×g for 10 minutes at 4° C. Cell lysates containing equal amounts of protein were resolved using sodium dodecyl sulfate-polyacrylamide gel electrophoresis, transferred to nitrocellulose membranes, and incubated with different primary antibodies. Protein expression was detected using a horseradish peroxidase-conjugated secondary antibody (Bio-Rad, Hercules, CA) and electrochemiluminescence reagent (Amersham Biosciences, Pittsburgh, PA). For the quantification of protein expression, the band intensities were measured using ImageJ, RRID:SCR_003070 (National Institutes of Health, Bethesda, MD) (Schneider, et al., Nature methods 2012; 9(7):671-5) and normalized first to β actin and then to its respective positive control (expression in HN31, UMSCC4, or C33A, as noted in the Figures).

### Example 3

### Apoptosis, Cell Cycle, and Senescence Assays

To measure apoptosis, TUNEL staining was performed with an APO-BRDU Kit (BD Biosciences, San Jose, CA) and annexin V/propidium iodide staining with an FITC Annexin V Apoptosis Detection Kit PE (eBioscience, San Diego, CA) as described previously (Kalu, et al., Cancer Lett 2018; 431:64-72). For the cell cycle analysis, cells were harvested, fixed, incorporated with bromodeoxyuridine (BrdU), and stained with 7-aminoactinomycin D using a BrdU Flow Kit (BD Biosciences, San Jose, CA). Data were acquired with a 3-laser, 10-color Gallios flow cytometer (Beckman Coulter, Brea, CA) and analyzed using Kaluza software, RRID:SCR_016182 (Beckman Coulter, Brea, CA). Assays were performed in triplicate, and each test was completed twice on different days. To measure senescence, cells were stained using the CellEvent Senescence Green Detection Kit (Thermofisher Scientific, Waltham, MA). Briefly, after drug treatment, cells were incubated with Senescence Green Probe, a fluorescence-based reagent that contains 2 galactoside moieties that make it specific to 0-galactosidase. The enzyme-cleaved product is retained within the cell, and it emits a fluorogenic signal that was measured at 490/514 nm using a flow cytometer.

### Example 4

### siRNA Transfection

c., Dallas, Tx) targeting E7, TRIP13, MAD2L1, and BUBIB or scrambled controls were transfected into cells using lipofectamine RNAi Max (ThermoFisher, Waltham, MA) in Opti-MEM (Invitrogen, Waltham, MA) according to the manufacturers' standard protocols. Cells were transfected with siRNA for 24 hours prior to the initiation of drug treatment.

### Example 5

### Generation of Stable Cells Using Lentiviral Infection

The lentiviral-based shRNA (GIPZ plasmid) used to knock down human RB1 expression was purchased from Horizon Discovery (Waterbeach, United Kingdom). On the basis of the knockdown efficiency of Rb1 protein expression in 3 HPV-negative cell lines (HN31, UMSCC4, and MDA88 6LN), shRblclones were selected (#2, #4, and #6) for this study. These were the clones with the greatest reductions in Rb1 protein expression. The mature antisense sequences were as follows: 5′-TAAAGATGTATCCTATATC-3′ (SEQ ID NO:1) (shRb1 #2), 5′-TTAACTGAAATGAAATCAC-3′ (SEQ ID NO:2) (shRb1 #4), and 5′-TAAGTTCACATGTCCTTTC-3′ (SEQ ID NO:3) (shRb1 #6). To generate lentivirus-expressing shRNA for RB1, HEK 293T cells were transfected with GIPZ-non-silence (for the vector control virus) and GIPZ-shRB1 with jetPEI transfection reagent (RRID:CVCL_0063). The medium was changed 24 hours after transfection and then collected every 48 hours thereafter. The collected lentivirus-containing medium was centrifuged and then filtered (0.45 μM). Cells were seeded at 50% confluence 12 hours before infection, and the medium was replaced with a medium containing lentivirus. After 24 hours, the medium was replaced and the infected cells were selected with 2.5 μg/mL puromycin (Sigma-Aldrich, St. Louis, MO).

### Example 6

### In Vivo Tumor Growth Study

The in vivo tumor growth study was performed in accordance with the Guide for the Care and Use of Laboratory Animals of the National Institutes of Health and approved by MD Anderson's Institutional Animal Care and Use Committee. The PDX models were generated as described previously (Peng, et al., J Transl Med 2013; 11:198), and subjected to qPCR as described to determine the HPV type (Chaturvedi, et al., The Journal of Infectious Diseases 2014; 210(3):441-7; Fakhry, et al, JAMA Oncol 2019; 5(7):985-92). Briefly, tumor tissue was cut into small fragments (5-6 mm) and implanted subcutaneously into the flanks of nude mice. The skin incisions were closed with skin clips that were removed after 10 to 15 days. Once tumors reached an average of 150 to 300 mm³, the mice were randomized into either vehicle group or alisertib group. Alisertib (Takeda Pharmaceuticals, Lexington, MA; 10 mg/kg in 10% β-cyclodextrin) was administered via oral gavage once daily on a weekly schedule of 6 days on and 1 day off. Tumors were monitored daily, and tumor volume (length×width²×0.5) was evaluated twice per week with digital calipers. Mice were euthanized when tumors reached 2000 mm³. The endpoint for the survival studies was the tumor burden. Survival was measured using the Kaplan-Meier method. Survival curve analysis was performed with GraphPad Prism software version 9 (RRID:SCR_002798).

### Example 7

### Genomic Data from TCGA Cohorts

Files containing raw RSEM count data from the TCGA RNA-seq datasets that were previously harmonized computationally and mapped through the Toil open source calling pipeline (Vivian, et al, Nat Biotechnol 2017; 35(4):314-6) were downloaded from Amazon web services (AWS) S3 as previously described. RSEM counts were normalized to get upper quartile FPKM values (FPKM-UQ) with an in-house script in R studio. Essentially, non-protein coding RNAs were removed using annotations from Ensembl, which left 19,345 genes. The 75^{th} percentile RSEM value (excluding zeros) for each TCGA sample was calculated and used to derive FPKM-UQ values. For most statistical analyses, the log 2 (FPKM-UQ+0.01) transformation was applied.

A total of 517 RNA-seq samples from HNSCC tumors were acquired (Nature 2015; 517(7536):576-82) using original TCGA marker approach. Detection of HPV infection and HPV integration and measurement of HPV gene expression in TCGA data sets were performed by applying the VirusSeq pipeline RRID:SCR_005206 (Chen, et al., Bioinformatics 2013; 29(2):266-7). Tumor HPV status was established by considering an empirical cutoff of 1000 read counts mapped against the viral genome (Gleber-Netto, et al., JCI Insight 2019; 4(1) doi 10.1172/jci.insight.124762).

### Example 8

### Statistical Analyses

The concordance correlation coefficient was computed to assess the reproducibility of the normalized growth rates of the 2 replicates. To determine whether drugs of the same target class cluster together because of similar drug potency, unsupervised drug clustering was performed based on AOC_LD values. In the clustering analysis, 6 distinct drug clusters were observed based on the clustering trees. A chi-square test was applied to compare drug classification and the cluster membership. The Fisher exact test was applied to compare mutations status (wild-type and mutant) and drugs' response data (sensitive and resistant). In these analyses, p≤0.05 was considered statistically significant.

To identify protein markers, RPPA protein expression levels were compared between drug-sensitive and -resistant groups using 2-sample t-tests. The Benjamini-Hochberg method was used to adjust for multiple hypothesis testing. An appropriate false discovery rate q value was used to identify protein markers significantly associated with drug-sensitive or -resistant groups. All statistical and bioinformatical analyses were performed using R (version 3.6.0).

### Example 9

### Landscape of Drugs that Cause Cell Death in Squamous Carcinoma

The effects of 864 unique drugs from 51 classes was tested in 16 HPV-positive and 17 HPV-negative HNSCC and cervical squamous cancer cell lines (Table 2) that were characterized previously (Kaiu, et al., Oncotarget 2017; 8(49):86369-83). Drugs without a well-defined target or with unique targets (i.e., targets not shared with other drugs in the screen) were categorized as “other.” As with previous HTDS (Kalu, et al., Cancer Lett 2018; 431:64-72), the quality control metrics showed that the data were reproducible.

TABLE 2
HNSCC and Cervical Cancer Cell Lines Tested in the Study.

| | Abbreviated | | HPV | Doubling times |
|---|---|---|---|---|
| Cell line | name | Tumor source | status | (days) |
| HN4 | | REC (L) | Negative | 1.63 |
| HN31 | | LN (HN30) | Negative | 0.88 |
| UM-SCC-1 | UM1 | REC (OC) | Negative | 1.75 |
| UM-SCC-4 | UM4 | OP | Negative | 1.48 |
| UM-SCC-6 | UM6 | OP | Negative | 2.44 |
| UM-SCC- | UM17B | EXT (UM- | Negative | 1.55 |
| 17B | | SCC-17A) | | |
| UM-SCC-19 | UM19 | OP | Negative | 2.47 |
| UM-SCC- | UM22A | HP | Negative | 2.08 |
| 22A | | | | |
| UM-SCC-25 | UM25 | LN (L) | Negative | 1.84 |
| UM-SCC-85 | UM85 | REC (nose) | Negative | 2.24 |
| MDA886LN | | LN (L) | Negative | 1.44 |
| PCI15B | | LN (PCI-15A) | Negative | 1.54 |
| FaDu | | HP | Negative | 0.65 |
| Detroit562 | | Pleural | Negative | 2.74 |
| | | effusion (P) | | |
| 183 | | OP | Negative | 3.21 |
| PCI13 | | OC | Negative | 1.62 |
| 93-VU-147T | VU-147T | OC | HPV16 | 2.27 |
| UD-SCC2 | UD2 | HP | HPV16 | 4.1 |
| UPCI: SCC- | UP090 | OC | HPV16 | 2.8 |
| 090 | | | | |
| UPCI: SCC- | UP152 | HP | HPV16 | 1.95 |
| 152 | | | | |
| UM SCC47 | UM47 | OC | HPV16 | 2.2 |
| UMSCC104 | UM104 | REC (OC) | HPV16 | 2.6 |
| UT-SCC-45 | UT45 | OC | HPV33 | 1.3 |
| HMS-001 | | OC | HPV16 | 5.79 |
| C33A | | Cervix | Negative | 1.36 |
| HT3 | | Cervix | Negative | 2.48 |
| C4 I | | Cervix | HPV18 | 3.99 |
| C4 II | | Cervix | HPV18 | 3.03 |
| CaSki | | Cervix | HPV16 | 3.21 |
| HeLa | | Cervix | HPV18 | 1.29 |
| ME180 | | Cervix | HPV68 | 1.48 |
| MS751 | | Cervix | HPV45 | 2.43 |
| SiHa | | Cervix | HPV16 | 2.63 |
| SW756 | | Cervix | HPV18 | 1.6 |

Because traditional methods of measuring drug sensitivity (e.g., calculating IC₅₀ values) can be confounded by the number of cell divisions during the course of an assay, an alternative method was adapted whose results are independent of the cell division rate (Hafner, et al., Nature methods 2016; 13(6):521-7). In this method, the area-over-the-curve lethal dose (AOC_LD) value is 0 when a drug does not kill cancer cells at any concentration and greater than 0 when cell death occurs; it has a maximum value of 1.

About half the drugs tested (n=425; 49%) had AOC_LD values of 0, and thus were ineffective, in all cell lines. The rest of the drugs (n=439; 51%) were potentially effective, as they led to cell death in at least one cell line. To determine if certain classes of drugs were more or less likely to lead to cell death, it was determined if drugs within each class were enriched in either the ineffective or effective categories by calculating the Pearson residual for each class. Most drug classes were enriched with either effective or ineffective drugs, providing evidence that drugs with the same targets had similar effects. Anthracyclines, vinca alkaloids, and all drugs targeting Aurora kinases, histone deacetylase (HDAC), microtubules, pyruvate dehydrogenase kinase 1 (PDK1), fibroblast growth factor receptors (FGFR), CDC7, Bruton Tyrosine Kinase (BTK1), or the proteasome were effective, whereas those targeting matrix metalloproteinase, nitrogen oxide synthetase, or poly (ADP-ribose) polymerase (PARP) were ineffective. To determine whether effective drugs within the same target class cluster together, unsupervised drug clustering of the effective drugs using their AOC_LD values was performed.

About half of the chemotherapy agents tested were effective (43/78; 55%). To further classify the effectiveness of these chemotherapy agents, they were into 7 categories based on their mechanisms. The taxanes, anthracyclines, topoisomerase inhibitors, and vinca alkaloids were almost universally effective, whereas most of the alkylating agents and platinum drugs were largely ineffective. Unsupervised clustering of the effective chemotherapy drugs according to their AOC_LD values demonstrated that drugs in the same category were more likely to cluster together than to cluster with drugs in a different class. Likewise, the drugs targeting the PI3K/AKT/mTOR pathway were divided into 7 categories based on their targets. Most mTOR and PDK1 inhibitors were effective, and the drugs in the other categories had variable efficacy. Drugs did not strongly cluster by target within the PI3K/AKT/mTOR group likely because of off-target effects and target overlap.

### Example 10

### Aurora Kinase Inhibitors are Effective Against HPV-positive Cancers

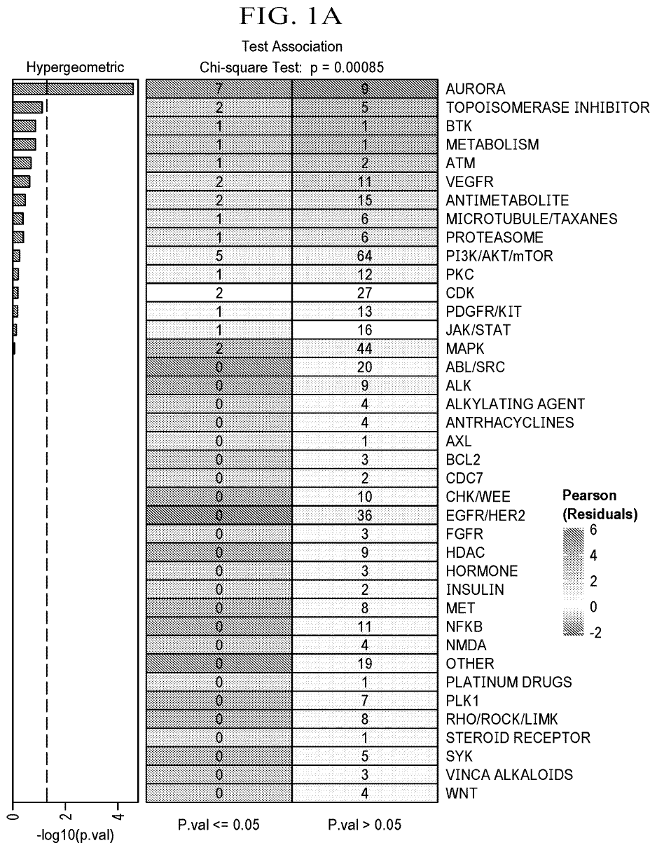

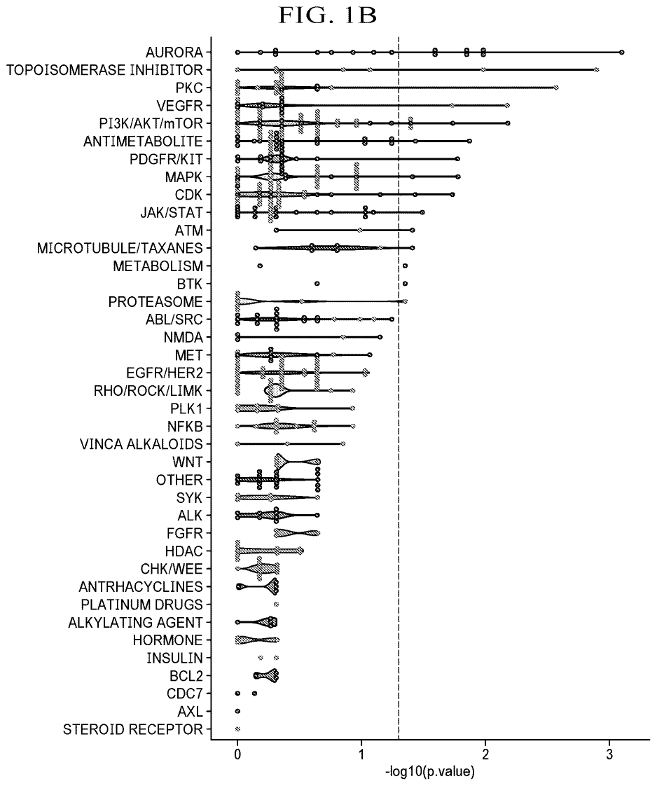

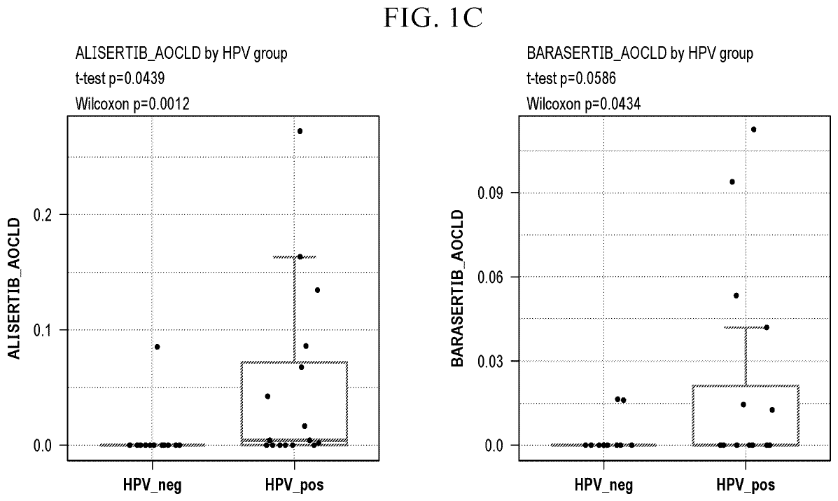

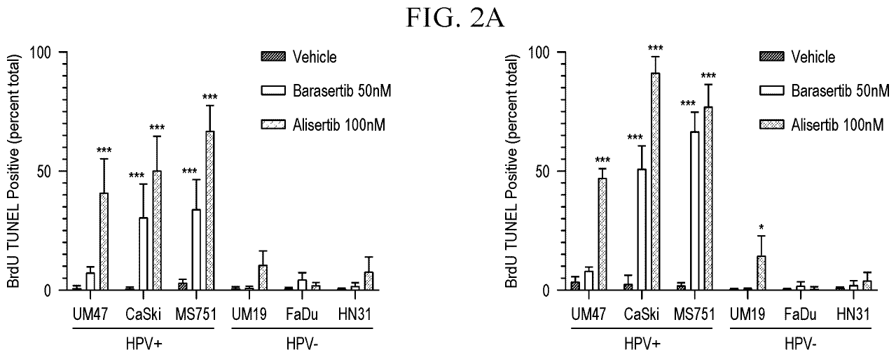

To determine if any drug or drug class was more effective than the others against HPV-driven cancers, the number of HPV-positive and -negative cell lines that were sensitive to the 439 effective drugs was compared to the number of HPV-positive and -negative cell lines that were resistant. Only 30 (7%) of the effective drugs had a differential effect based on HPV status, and only one class of drugs, the Aurora kinase inhibitors, was significantly more effective in HPV-positive cell lines (P<0.05, Fisher exact test; FIG. 1A, FIG. 1B).

Drugs from 15 classes were more effective in HPV-positive cell lines than in HPV-negative cell lines (Table 3). All the effective drugs in each of these 15 classes were closely examined to determine if there was a trend for a class effect that warranted further investigation. Drugs from 2 of these classes (antimetabolites and MAPK inhibitors) had variable effects, with some drugs more effective in HPV-negative cell lines and others more effective in HPV-positive cell lines. Most drug classes (antimetabolites and inhibitors of CDK, PKC, PDGFR/KIT, MAPK, proteasome, JAK/STAT, PI3K/AKT/MTOR, topoisomerase, or VEGFR) did not demonstrate a class effect, as a minority of the drugs in these classes demonstrated a differential effect based on HPV status, and the direction of the effect was variable within the class. Several drug classes (BTK, ATM, and metabolism inhibitors) were not studied further because of their overall lack of class efficacy. Within the large family of PI3K/AKT/MTOR inhibitors, several mTOR inhibitors were more effective in HPV-positive cell lines than in HPV-negative ones, but the overall efficacy of these drugs was low with the exception of Torin-2, which has several off-target effects at a concentration of 3.16 μM. The more specific mTOR inhibitors—everolimus, rapamycin, and ridaforolimus—caused cell death only in a single cell line at concentrations known to inhibit mTOR. Likewise, drugs affecting microtubules were consistently more effective in HPV-positive cell lines, but in contrast to the mTOR inhibitors, every drug in the microtubule/taxane class caused cell death in most of the cell lines tested, including the HPV-negative cell lines. Only one microtubule/taxane was significantly more effective in HPV-positive than in HPV-negative cell lines. Consistent with these findings, taxanes have been shown to have broad clinical efficacy in squamous cancers.

TABLE 3
List of Drugs from 15 Classes were More Effective in HPV-
positive Cell Lines than in HPV-negative Cell Lines.

| | HPV | | HPV | | | | |
|---|---|---|---|---|---|---|---|
| | negative | | positive | | odds | | |
| Drug | Resistant | Sensitive | Resistant | Sensitive | ratio | p value | Classification |
| ALISERTIB | 16 | 1 | 6 | 10 | 23.71 | 0.0008 | AURORA |
| AMSACRINE | 15 | 2 | 5 | 11 | 14.80 | 0.0013 | TOPOISOMERASE |
| | | | | | | | INHIBITOR |
| 365250 | 17 | 0 | 9 | 7 | Inf | 0.0027 | PKC |
| BX912 | 16 | 1 | 8 | 8 | 14.66 | 0.0066 | PI3K/AKT/mTOR |
| AXITINIB | 16 | 1 | 8 | 8 | 14.66 | 0.0066 | VEGFR |
| CYC116 | 15 | 2 | 7 | 9 | 8.92 | 0.0104 | AURORA |
| TOZASERTIB | 15 | 2 | 7 | 9 | 8.92 | 0.0104 | AURORA |
| ETOPOSIDE | 15 | 2 | 7 | 9 | 8.92 | 0.0104 | TOPOISOMERASE |
| | | | | | | | INHIBITOR |
| FLUDARABINE | 6 | 11 | 13 | 3 | 0.14 | 0.0134 | ANTIMETABOLITE |
| ZM447439 | 14 | 3 | 6 | 10 | 7.24 | 0.0134 | AURORA |
| GSK1070916 | 12 | 5 | 4 | 12 | 6.71 | 0.0149 | AURORA |
| 521234 | 16 | 1 | 9 | 7 | 11.53 | 0.0167 | PDGFR/KIT |
| 559396 | 9 | 8 | 15 | 1 | 0.08 | 0.0167 | MAPK |
| 217699 | 17 | 0 | 11 | 5 | Inf | 0.0184 | CDK |
| AZD8055 | 17 | 0 | 11 | 5 | Inf | 0.0184 | PI3K/AKT/mTOR |
| NINTEDANIB | 17 | 0 | 11 | 5 | Inf | 0.0184 | VEGFR |
| PF03814735 | 15 | 2 | 8 | 8 | 7.02 | 0.0255 | AURORA |
| SNS314 | 15 | 2 | 8 | 8 | 7.02 | 0.0255 | AURORA |
| AT9283 | 10 | 7 | 3 | 13 | 5.82 | 0.0324 | JAK/STAT |
| JNJ7706621 | 11 | 6 | 4 | 12 | 5.19 | 0.0366 | CDK |
| BLEOMYCIN | 13 | 4 | 6 | 10 | 5.12 | 0.0366 | ANTIMETABOLITE |
| 361555 | 13 | 4 | 6 | 10 | 5.12 | 0.0366 | PI3K/AKT/mTOR |
| 118501 | 16 | 1 | 10 | 6 | 8.98 | 0.0391 | ATM |
| DOVITINIB | 16 | 1 | 10 | 6 | 8.98 | 0.0391 | MAPK |
| WYE354 | 16 | 1 | 10 | 6 | 8.98 | 0.0391 | PI3K/AKT/mTOR |
| COLCHICINE | 7 | 10 | 1 | 15 | 9.81 | 0.0391 | MICROTUBULE/ |
| | | | | | | | TAXANES |
| AT7867 | 17 | 0 | 12 | 4 | Inf | 0.0445 | PI3K/AKT/mTOR |
| IBRUTINIB | 17 | 0 | 12 | 4 | Inf | 0.0445 | BTK |
| KT5720 | 17 | 0 | 12 | 4 | Inf | 0.0445 | METABOLISM |
| CARFILZOMIB | 5 | 12 | 0 | 16 | Inf | 0.0445 | PROTEASOME |

All 16 Aurora kinase inhibitors (AMG900, MLN8054, CCT129202, CCT137690, CYC116, MK5108, alisertib, PF03814735, PHA680632, danusertib, TAK901, tozasertib, ZM447439, barasertib, GSK1070916, and SNS314) tended to be more effective in HPV-positive cell lines, and this difference was statistically significant for 7 of them using AOC_LD as a dichotomous variable (FIG. 1C). Three other drugs in the HTDS also inhibited Aurora kinases: the JAK3 inhibitor AT9283, the CDK2 inhibitor JNJ7706621, and hesperidin, which inhibits multiple targets. All 3 drugs were more effective in HPV-positive than in HPV-negative cell lines. Likewise, when AOC_LD was used as a continuous variable, the difference was statistically significant (P<0.05, Wilcoxon test) for 7 of the Aurora kinase inhibitors and for hesperidin, AT9283, and JNJ7706621. In addition to the AOC_LD values, the ICso values were computed for all 16 Aurora kinase inhibitors in HPV-positive cell lines. Similar to the AOC_LD metric, most ICso values for Aurora kinase inhibitors are low in HPV-positive cell lines.

### Example 11

### Aurora Kinase Inhibitors Cause Apoptosis in HPV-Positive Cancer Cells

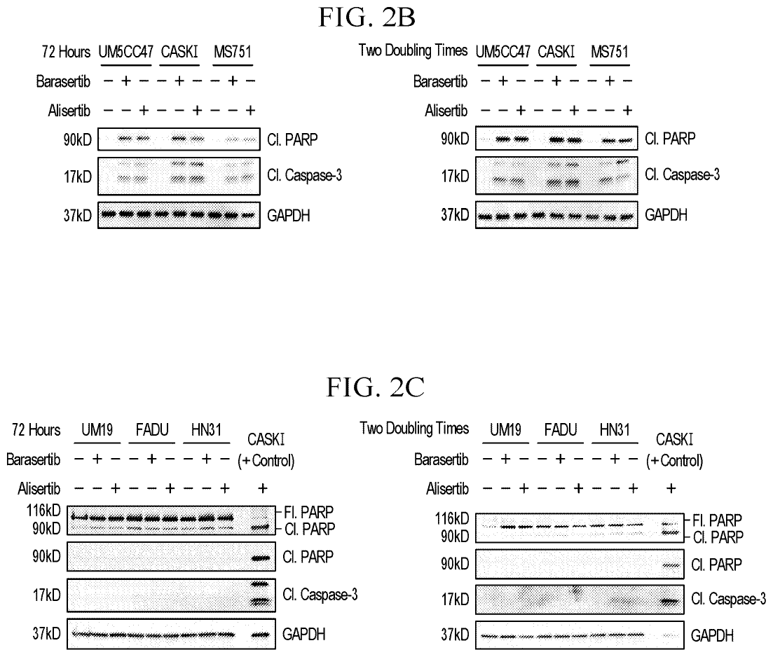

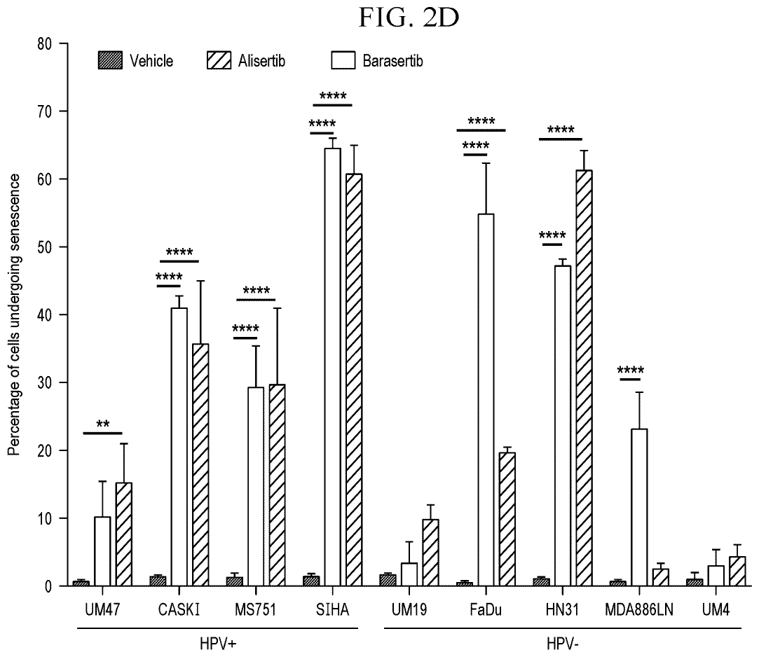

To confirm that Aurora kinase inhibition led to cell death in HPV-positive cell lines, the effect of 2 clinically relevant and specific drugs, alisertib and barasertib, on apoptosis was examined using 3 orthogonal methods and relevant drug concentrations. In addition to using a standard treatment time of 72 hours, the cells were also treated for 2 doubling times to overcome the artifact of a fixed treatment time for cell cycle-specific drugs in cell lines with diverse cycling times. Terminal deoxynucleotidyl transferase dUTP nick end labeling (TUNEL)-positive apoptotic nuclei significantly increased in HPV-positive cancer cell lines (UMSCC47, CaSki, and MS751) treated for 72 hours or 2 doubling times. However, no appreciable levels of apoptosis were noted in matched HPV-negative cancer cell lines (UMSCC19, FaDu, and HN31; FIG. 2A). Likewise, apoptosis induced by barasertib or alisertib was associated with selective caspase-3 activation and PARP cleavage in HPV-positive cancer cell lines (FIGS. 2B, C). In contrast, no significant differences in senescence induced by Aurora kinase inhibitors were noted between HPV-positive cells and HPV-negative cells (FIG. 2D).

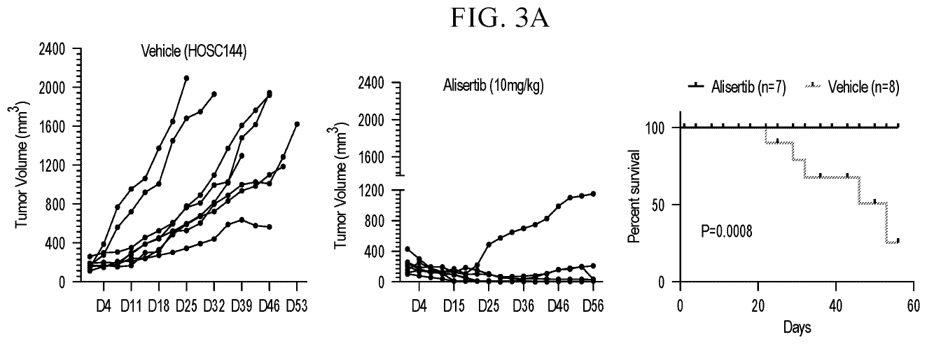

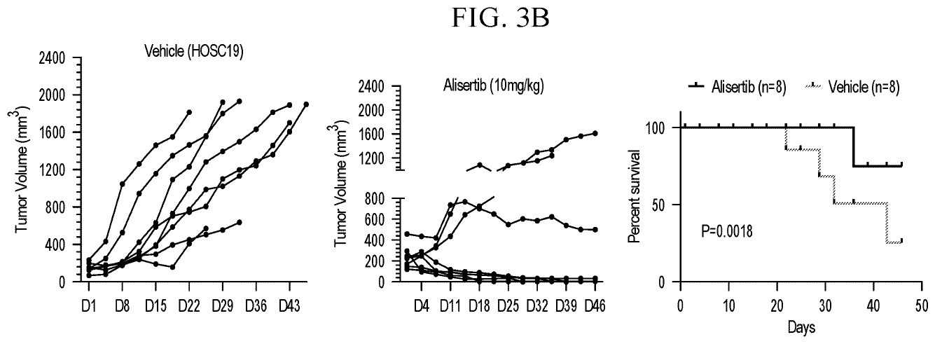

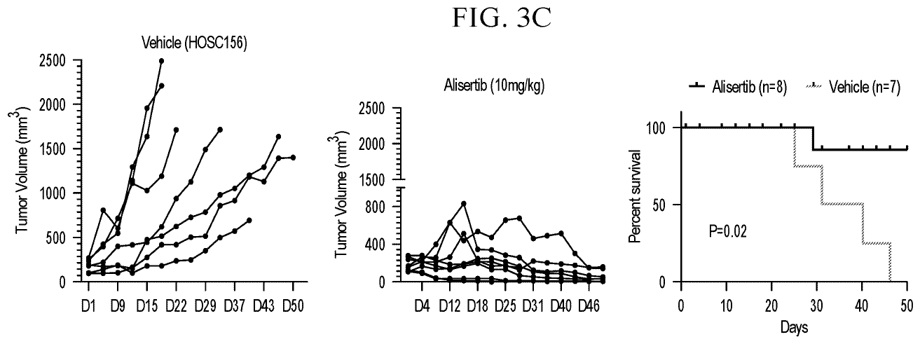

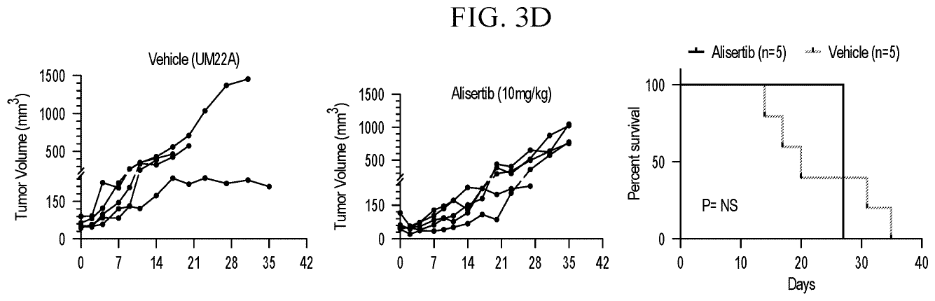

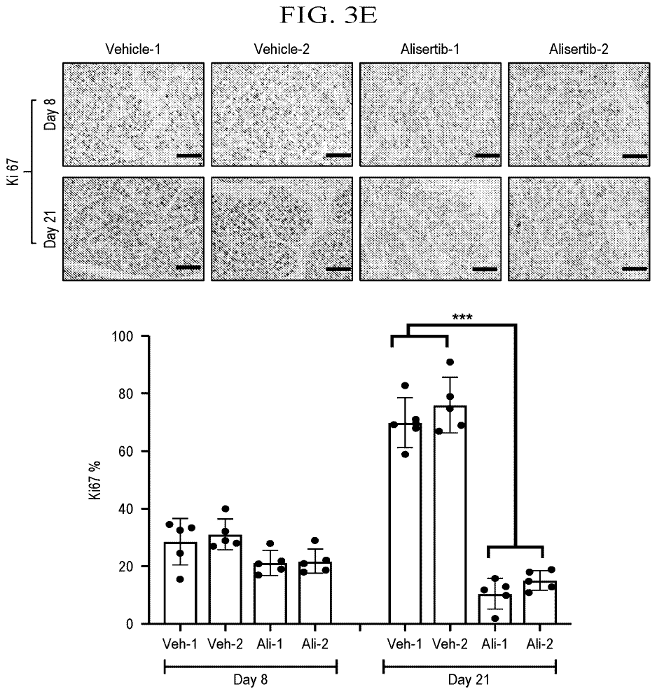

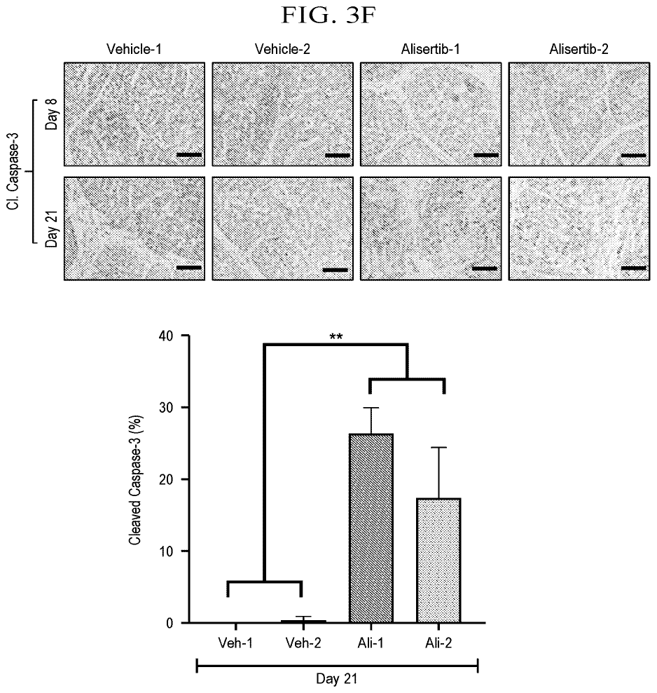

To test the effect of Aurora kinase inhibition in vivo, three patient-derived xenograft (PDX) models that were derived from patients with p16-positive oropharynx cancer [HOSC144 (HPV16, HPV52), HOSC19 (HPV18, HPV59), and HOSC 156 (HPV18)], were implanted in nude mice. A low dose of alisertib (10 mg/kg) was administered continuously for 3 weeks. In agreement with the in vitro findings, clear evidence of tumor regression was observed in both PDX models. This anti-tumor effect was accompanied by a concordant increase in survival duration (FIGS. 3A-C). In addition, an HPV-negative cell line with high Rb protein levels was also tested and it was observed that it was resistant to alisertib treatment (FIG. 3D). Also, a sustained response was observed even after treatment was stopped. Tumors collected from alisertib-treated mice on days 8 and 21 had significantly lower levels of Ki67 staining and increased levels of cleaved caspase-3 compared to those from vehicle-treated mice (FIGS. 3E, F).

### Example 12

### Rb Expression and TP53 Mutations Predict Cell Response to Aurora Kinase Inhibitors

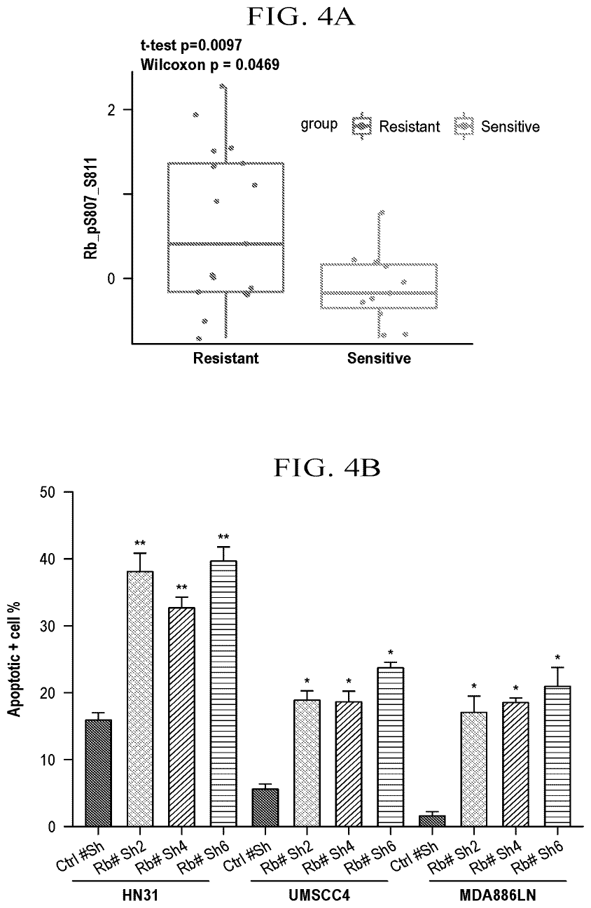

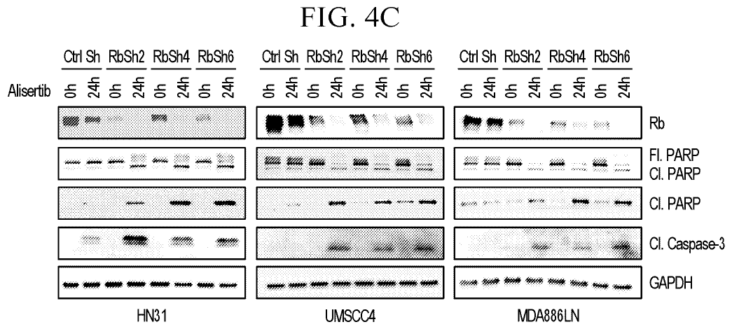

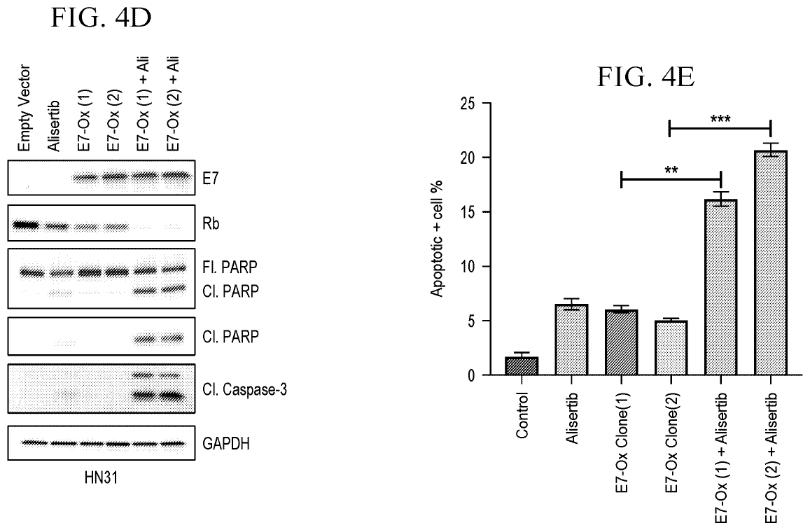

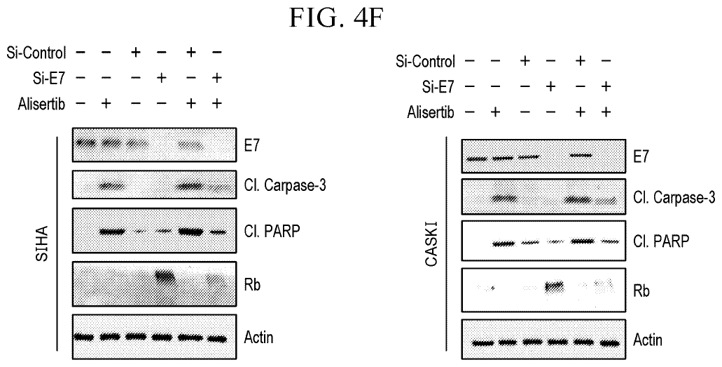

To determine if factors other than HPV status predict response to Aurora kinase inhibitors, protein expression was compared using Reverse Phase Protein Arrays (RPPA) and gene mutation using whole exome sequencing (WES) to drug efficacy using AOC_LD. The expression levels of 19 proteins were correlated with sensitivity to at least 4 Aurora kinase inhibitors (ACC_pS79, Annexin VII, beclin, cMet, ERCC1, FoxM1, HSP27, MEK1_pS217_S221, p21, PKC.delta_pS664, PMS2, Rb_pS807_S811, SF2, Smad4, SOD1, Tau, transglutaminase, ULK1_pS757, and X143.3.epsilon) after correction for multiple testing (P<0.05, adjusted for the false discovery rate). Immunoblotting was used to validate the RPPA findings in all 33 cells lines. Among the 19 proteins, the 8 were focused on that correlated with sensitivity to the most drugs and in which the correlations were consistently in a single direction (i.e., expression predicted sensitivity or resistance but not both). On the basis of published studies (Gong, et al., Cancer Discov 2019; 9(2):248-63; Oser, et al., Cancer Discov 2019; 9(2):230-47), immunoblotting was used to explore 7 additional candidate proteins that were not in the RPPA. Of the 15 chosen proteins, distinct immunoblotting bands were detected for all except Tau. Only Rb, pRb (S807/811), and p16 were consistently correlated with drug sensitivity (P<0.05 per the Wilcoxon test; FIG. 4A, and some data not shown). Specifically, the expression of pRb (S807/811) determined using RPPAs or immunoblotting, total Rb determined using immunoblotting, and p16 (CDKN2A) determined using immunoblotting were correlated with the efficacy of all the Aurora kinase inhibitors, with statistical significance in 6, 6, 10, and 7 of the drugs, respectively. In addition, the pRb levels were compared with sensitivity (AOC_LD) to 15 Aurora kinase inhibitors in only the HPV-positive cell lines. pRb (S807/811) expression is consistently lower in sensitive cell lines.

The extent to which drug sensitivity was correlated with 50 of the most common gene mutations in HNSCC (Nature 2015; 517(7536):576-82) was determined. Only 35 of the 50 gene mutations were found in 2 or more cell lines in the HTDS. Of those 35 gene mutations, only TP53 mutations were correlated with resistance to 7 Aurora kinase inhibitors. Aurora B mRNA, Aurora B protein, and Aurora A mRNA expression did not correlate with Aurora kinase inhibitor efficacy.

### Example 13

### Manipulation of RB1 Affects Aurora Kinase Inhibition-Induced Apoptosis in Squamous Cancers

To determine if Rb protein expression is instrumental in Aurora kinase inhibitor-induced apoptosis, three independent methods were used to alter Rb expression in HPV-negative squamous cancer cell lines. First, HPV-negative cells were treated with alisertib and depleted the RB1 levels using transient transfection. Rb depleted HPV-negative cells showed an increase in cleaved PARP levels upon treatment with alisertib. Next, a more stable system was employed to knock down RB1 using short hairpin RNA (shRNA) that significantly reduced Rb protein expression in 3 HPV-negative cell lines. Alisertib led to an approximately 3-fold increase in annexin V-positive apoptotic cells when Rb was depleted (FIG. 4B). Additionally, alisertib alone did not induce apoptosis in control cells with detectable Rb protein levels. However, alisertib induced significant apoptosis in those same cell lines with reduced Rb levels, as evidenced by cleaved caspase-3 and cleaved PARP (FIG. 4C). To reduce Rb protein levels using an independent method that recapitulated the HPV phenotype, a stable plasmid to induce E7 expression was used. This method caused Rb protein degradation in 2 HPV-negative cell lines. As expected, the RB1 RNA levels remained unchanged. Alisertib treatment selectively enhanced apoptosis in cells overexpressing E7, as indicated by cleaved caspase-3 and PARP levels (FIG. 4D) and annexin V (FIG. 4E).

The effect of RB1 silencing on the expression of other RB family members (RBL1, RBL2) was also examined. It was observed that the levels of RBL1 remained unchanged with the treatment. However, the protein levels of RBL2 decreased with RB1 silencing as well as with alisertib treatment. The effect of RB1 silencing on RBL2 was likely due to pooled siRNAs. Using RB1 shRNA, an effect of RB1 depletion on RBL2 was not observed, but the enhanced alisertib-induced apoptosis was observed, supporting a role for RB1 alone in this effect.

A decrease of Rb protein expression following Aurora kinase inhibition was noted, that may be related to G2/M cell cycle arrest. Alternatively, it may be that the HPV negative cells with low Rb expression are the ones that survive after Aurora inhibition. To test this at the single cell level, two HPV-negative cell lines were co-stained with anti-Rb labelled with FITC conjugate and anti-cleaved caspase-3 labelled with TRITC and treated with alisertib or vehicle control. As expected in these unsynchronized cells, Rb expression was detectable, but modestly heterogeneous in control cells. Consistent with the Western blots, Rb expression was notably lower and cleaved caspase 3 slightly higher in the alisertib-treated cells than in control cells. Consistent with prior data, cells with higher Rb levels have no or very low levels of cleaved caspase-3. Cells with lower Rb levels have modest levels of cleaved caspase-3.

It was hypothesized that increased Rb expression can rescue HPV-positive cells from Aurora kinase inhibition-induced apoptosis. Inducing Rb expression using a stable or inducible plasmid was not successful because the existing E7 led to Rb protein degradation. To overcome this problem, E7 expression was knocked down using small interfering RNA (siRNA), which resulted in a 3-fold increase in Rb protein levels. As expected, alisertib treatment caused apoptosis in the HPV-positive cell lines but not in the same cells with increased Rb expression (FIG. 4F).

### Example 14

### Aurora Kinase Inhibition-Induced Apoptosis Depends Upon a Balance of Mitotic Checkpoint Proteins in Rb-Deficient Squamous Cancers

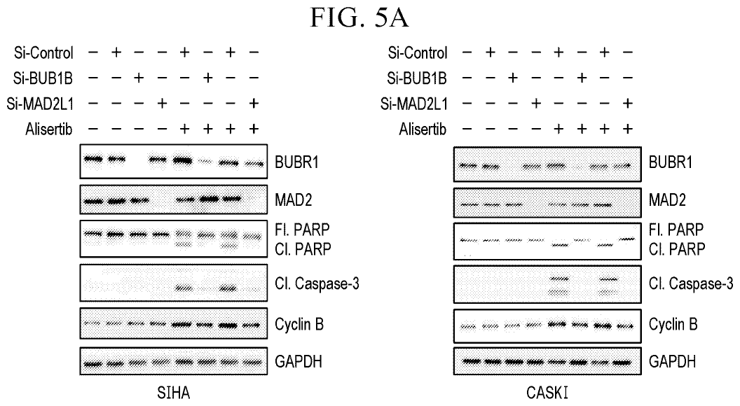

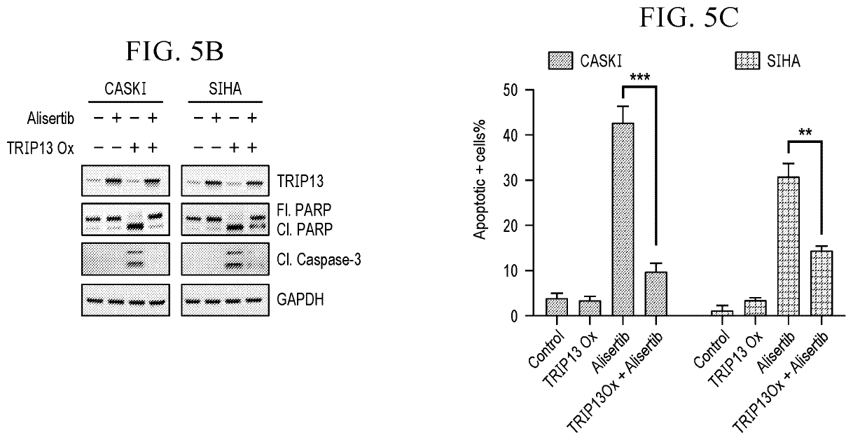

In addition to having a well-characterized role in controlling entry into S phase, the inhibition of the Rb tumor suppressor pathway and subsequent activation of E2F directly enhance the expression of the mitotic checkpoint gene Mad2 and prolong mitosis. MAD2L1 (Mad2) has a core E2F transcription factor binding site in its promoter and Rb-deficient cancer cells have elevated levels of Mad2. The primed mitotic checkpoint complex (MCC) may create an Achilles heel for cancer cells lacking Rb because these cells depend on Aurora kinase activity to promote mitotic exit and survival. To test the hypothesis that Mad2 and BUB1B mediate Aurora kinase inhibitor sensitivity, siRNA was used to deplete their levels in 2 HPV-positive cell lines. In both cell lines, reduced levels of Mad2 or BU B1B rescued the cells from alisertib-induced apoptosis (FIG. 5A).

In normal cells, prolonged mitosis resulting from Mad2 overexpression can lead to G1 arrest or mitotic cell death, so it is unclear why Mad2 overexpression is tolerated in HPV-positive cancers. One potential mechanism to prevent stalled mitosis is the regulation of Mad2 function via TRIP13, It was hypothesized that HPV-positive cells have an increased dependency on TRIP13 for their mitotic exit. Therefore, the transient overexpression of TRIP13 was induced in 2 HPV-positive cell lines and a partial rescue was observed from alisertib-induced apoptosis as indicated by PARP and caspase-3 cleavage (FIG. 5B) and annexin V expression (FIG. 5C). Similarly, TRIP13 overexpression in 2 HPV-negative cell lines with RB1 knockdown (done with shRNA) reduced apoptosis caused by Aurora kinase inhibition (FIG. 5D).

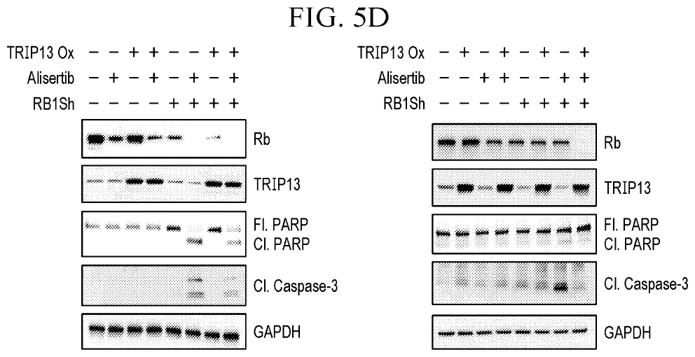

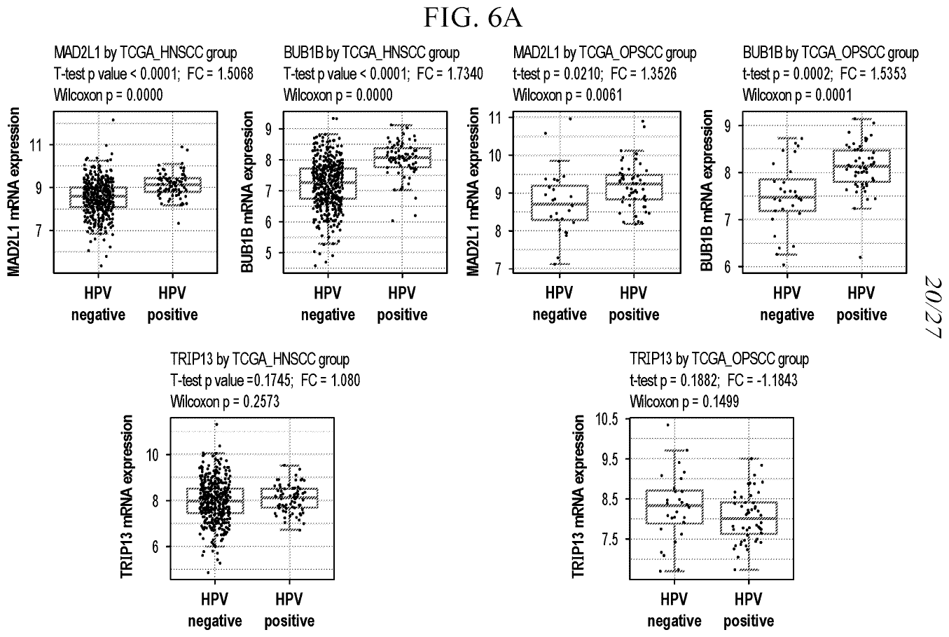

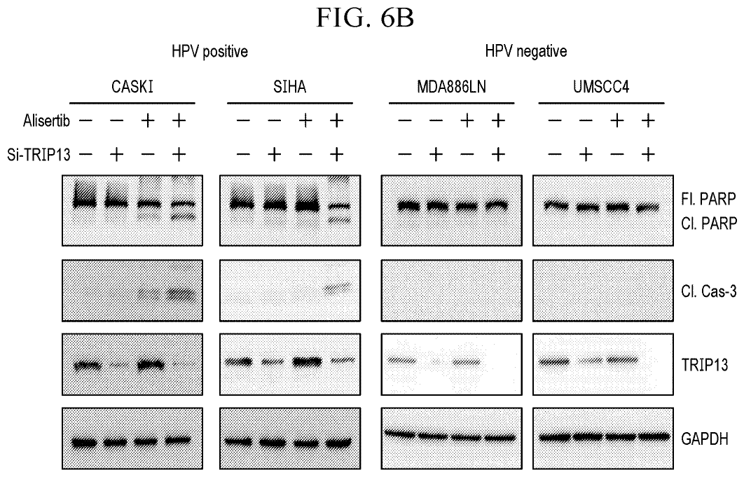

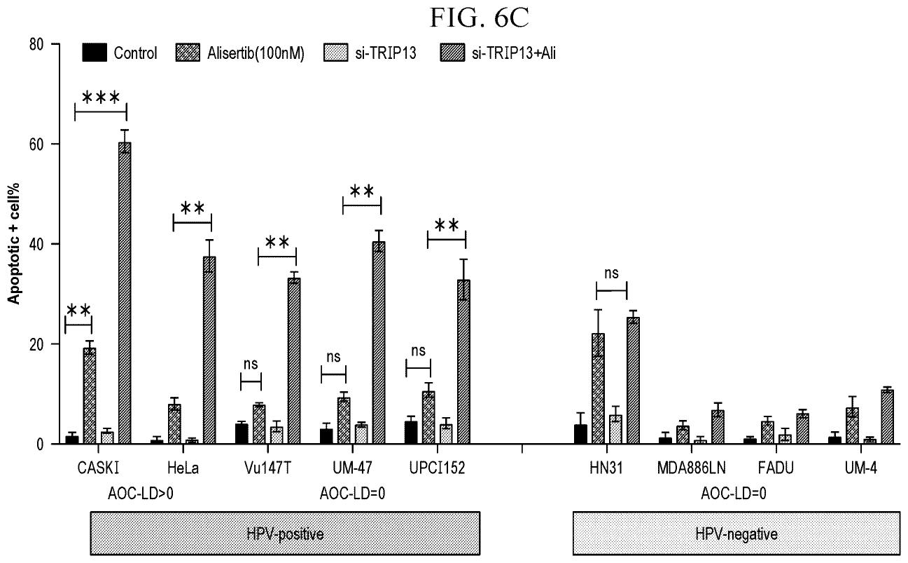

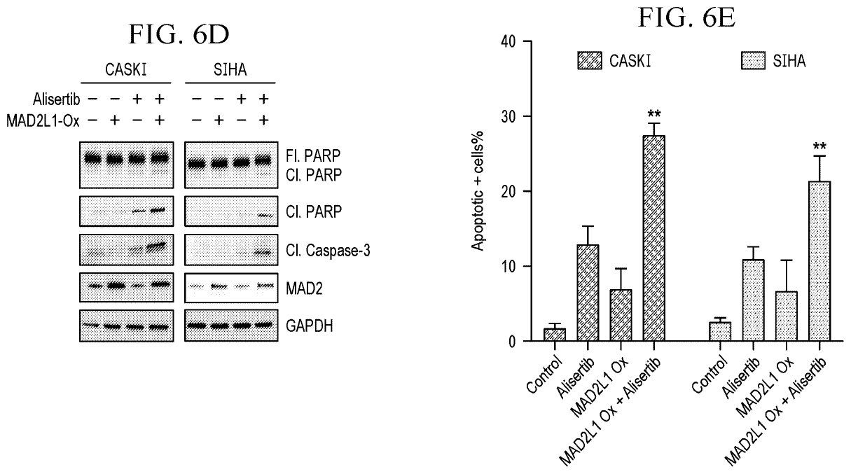

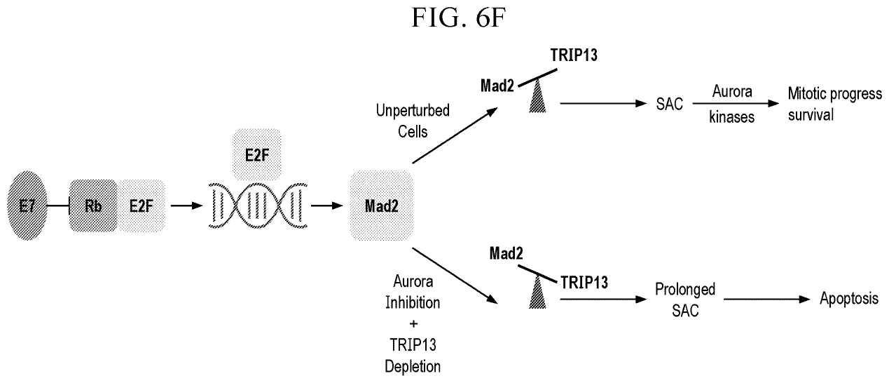

MCC gene expression has never been examined in HPV-positive tumors. To determine if MCC genes are overexpressed in HPV-driven tumors, their expression was examined in tumor samples from 517 HNSCC patients in The Cancer Genome Atlas (TCGA) (Nature 2015; 517(7536):576-82). mRNA expression was compared between 70 HPV-positive and 447 HPV-negative tumors and found significantly higher expression of both BUB1B (BUBR1, BUB1β) and MAD2L1 (Mad2) in HPV-positive tumors (P<0.0001; FIG. 6A). Only HPV-positive (n=51) and HPV-negative oropharynx squamous cell carcinoma (OPSCC, n=28) was compared and a significantly higher expression of both BUB1B and MAD2L1 in HPV-positive OPSCC tumors was found, P<0.05. (FIG. 6A, right panel). The expression of TRIP13 was lower in HPV positive than in HPV negative OPSCC, but the difference was not significant in both the sample sets.

Next, it was determined if TRIP13, MAD2L1, and BUB1B expression predicted response to Aurora kinase inhibition in HPV-positive cancer cells. The mRNA expression of TRIP13, MAD2L1, and BUBIB was compared to Aurora kinase inhibitor sensitivity. Consistent with the model, higher levels of TRIP13 and lower levels of BUBIB predicted resistance.

### Example 15

### TRIP13 Knockdown Enhances Aurora Kinase Inhibition-Induced Cell Death in HPV-Positive Squamous Cancers

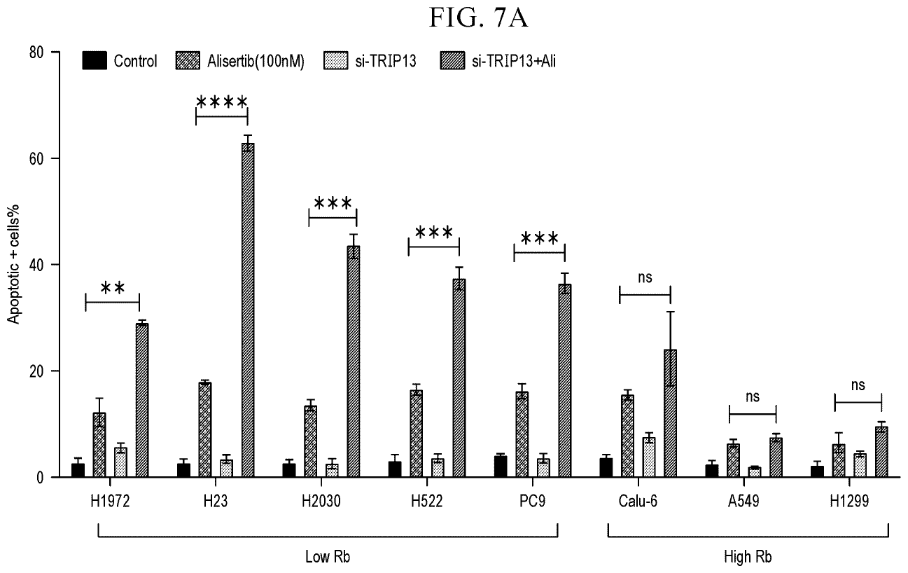

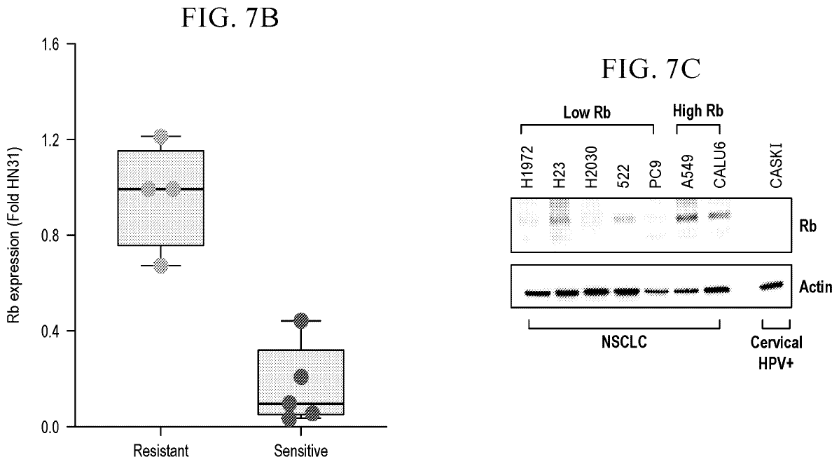

TRIP13 is an enzyme that is potentially a therapeutic target. To determine if Aurora kinase and TRIP13 inhibition have synthetic lethality in HPV-positive cancers, first transfected HPV-positive cells were transfected with two different siRNAs against TRIP13 that reduced the expression of the TRIP13 mRNA and protein levels. Densitometric analysis of the band intensities showed that both siRNAs decreased TRIP13 expression by more than 50% (**, P<0.001) compared to control siRNA. Next, both HPV-positive and HPV-negative cell lines were transfected with TRIP13 or control siRNA and treated them with low concentrations of alisertib. TRIP13 knockdown alone did not cause apoptosis, consistent with its effect in other cancer and normal cells. Higher levels of cleaved caspase-3 and cleaved PARP levels were observed in HPV-positive cells transfected with TRIP13-knockdown with alisertib than in control cells treated with alisertib, whereas no expression of caspase-3 activation and PARP cleavage were observed in HPV-negative cells (FIG. 6B). In addition, the combination of TRIP13 knockdown with alisertib killed more than half of the cells from five HPV-positive cell lines without significantly affecting HPV-negative squamous cancer cells with wild type Rb (FIG. 6C) demonstrating selectivity. In addition, it was also observed that the combination led to significant apoptosis in HPV positive cell lines that were resistant to alisertib (i.e., AOC-LD=0 in the HTDS) supporting the model (FIG. 6C). To determine if the model was applicable to other cancers with Rb pathway defects, the combination of TRIP13 depletion and Aurora kinase inhibition was tested in non-small cell lung cancer (NSCLC) cell lines with various levels of Rb expression. It was found that cancer cells with low Rb levels experienced significantly more apoptosis with the combination than those with high Rb expression (FIG. 7. A-C).

Next, Mad2 overexpression was induced in 2 HPV-positive cell lines to test if altering the balance between TRIP13 and Mad2 would affect cells' sensitivity to Aurora kinase inhibition. Mad2 overexpression alone did not activate the apoptotic pathways. However, Mad2 overexpression combined with alisertib enhanced apoptosis (FIGS. 6D, 6E).

This study provides strong evidence to support a model in which HPV positive cancer cells maintain a balance of Mad2 and TRIP13 allowing mitotic exit and survival despite Rb loss. The combined inhibition of TRIP13 and Aurora kinase disrupts the balance and leads to enhanced cell death in Rb-deficient HPV-positive cancer cells (FIG. 6F).

### Example 16

### TRIP13 Inhibition Enhances Aurora Kinase Inhibition-Induced Cell Death in HPV-Positive Squamous Cancers

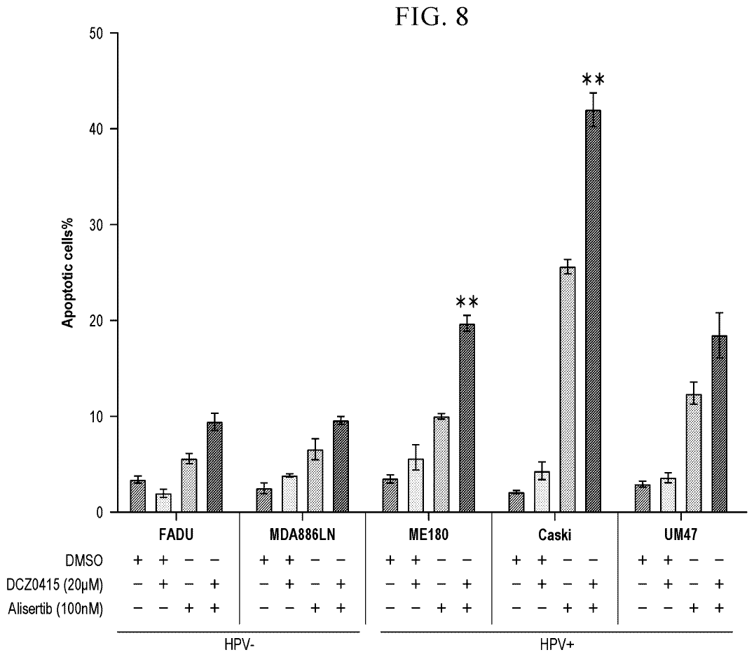

To determine whether TRIP13 inhibition with the small molecule inhibitor DCZ0415 enhances Aurora kinase inhibition-induced cell death in HPV-positive squamous cancers, the combined effect of DCZ0415 and alisertib was examined in two HPV-negative (FADU and MDA886LN) and three HPV-positive (ME180, Caski, UM47) cell lines. Cells were treated with 100 nmol/L alisertib and/or 20 mol/L DCZ0415 before annexin V-PE/7AAD staining and analysis with flow cytometry (FIG. 8). The bar graph represents the ratio of apoptosis (late plus early) among different groups. Values are the means±the standard deviations for 3 independent experiments. *P<0.05; **P<0.005; unpaired, 2-tailed Student t-test. As shown in FIG. 8, the combination of DCZ0415 and alisertib significantly increases apoptosis in HPV-positive, but not HPV-negative cell lines.

The current disclosure identifies, validates, and characterizes HPV status-selective therapy. The ability of 864 diverse compounds to cause cell death in a large panel of HPV-positive and -negative squamous cancer cells was tested. Aurora kinase inhibitors were identified as the drug class that was consistently more effective against HPV-positive cancers than HPV-negative cancers. Aurora kinase inhibition reduced tumor volume in vivo without toxicity. In addition to HPV status, baseline protein levels of Rb predicted the response to Aurora kinase inhibitors. Manipulating Rb expression altered sensitivity to Aurora kinase inhibitors. Further, the data identify a novel mechanism in which HPV-positive cancer cells maintain a balance of Mad2 and TRIP13 levels, allowing mitotic exit and survival despite Rb loss and Aurora kinase inhibition. Altering that balance enhanced Aurora kinase inhibition-induced apoptosis. Consistent with this observation, it was found that a combination of TRIP13 knockdown or inhibition with a small molecule with Aurora kinase inhibition led to marked apoptosis in HPV-positive cells, but not in HPV-negative cells that express Rb.

The in vivo data from three, independent PDX models of HPV-driven HNSCC demonstrated not only a striking tumor regression and survival duration benefit with Aurora kinase inhibition but also a sustained response even after treatment was stopped.

In the present study, it was determined that low Rb levels drive sensitivity to Aurora kinase inhibitors. The findings are consistent with those showing that other cancer types with RB1 loss-of-function mutations depend on mitotic kinases—particularly Aurora B and Aurora A—for survival. The inhibition of Rb pathways upregulates MCC genes and can result in chromosomal instability and prolonged mitosis. It was found that in HNSCC patients, BUB1B and MAD2L1 expression was higher in HPV-positive than in HPV-negative tumors, and that that the knockdown of Mad2 and BUB1B markedly reduced alisertib-induced apoptosis in HPV-positive squamous cancer cells.

To further investigate the mechanism of sensitivity to Aurora kinase inhibition and identify a potentially druggable target, the role of TRIP13 in HPV-positive cells was investigated. TRIP13 belongs to the AAA-ATPase family of chaperone proteins, which regulates the SAC by remodeling its effector, Mad2, from a “closed” (active) to an “open” (inactive) form during mitosis. TRIP13 localizes to kinetochores, and its knockdown affects the ATPase activity that delays metaphase-to-anaphase transition. Cancer cells overexpressing Mad2 have an increased dependency on TRIP13 for their mitotic exit. Given that Mad2 is overexpressed in HPV-positive patient tumors, it was hypothesized that the balance of Mad2 and TRIP13 dictates HPV-positive cancer cells' dependence on Aurora kinases for their mitotic exit and survival. In the present study, TRIP13 knockdown alone did not cause any significant cell death. However, the combined inhibition of TRIP13 and Aurora kinase surprisingly had a synergistic effect and caused marked apoptosis. Interestingly, Mad2 overexpression enhanced alisertib-induced apoptosis to a lesser degree than did TRIP13 depletion, leading us to speculate that endogenous TRIP13 levels inactivate Mad2 to protect cells. This finding emphasized that Mad2 overexpression increases cells' dependency on TRIP13 for survival. In Rb-proficient cancers, disrupting mitosis may also affect the efficacy of Aurora kinase inhibition.

Researchers and clinicians can take advantage of this relationship as a potential way to effectively target HPV-positive squamous cancer cells while sparing normal cells that express Rb and have unaltered levels of Mad2. The reduction of TRIP13 in normal cells causes only a mild mitotic delay without cell death. It was found that, in HPV-positive cancer cells with high endogenous levels of Mad2, combined TRIP13 and Aurora kinase inhibition led to striking levels of apoptosis. This result demonstrates that TRIP13 could be therapeutically targeted, with potentially low toxicity, in tumors with high Mad2 levels. Given that TRIP13 is an AAA-ATPase, it is a more readily druggable target than Mad2 is. Other AAA-ATPases, such p97 and ATAD2, are clinically important targets in cancer and have been targeted with small molecules that have shown potential promise in many therapeutic regimens.

TRIP13 also functions in DNA repair by promoting nonhomologous end joining (NHEJ) and inducing treatment resistance by binding to the NHEJ proteins KU70, KU80, and DNA-PKcs in head and neck cancer. Studies have also suggested that the inhibition of Aurora kinases stimulates the error-prone NHEJ repair of DNA double-strand breaks with increased DNA-PKcs phosphorylation. Thus, to examine the synergistic effects of TRIP13 and Aurora kinase inhibition, TRIP13 expression was knocked down in cells and the cells were treated with alisertib, or cells were treated with a combination of DCZ0415 and alisertib. TRIP13 inhibition enhanced the cytotoxic effects of alisertib in HPV-positive cells, which demonstrates that TRIP13 inhibition, in combination with other therapeutics, can reduce cancer cell survival.

Several other biomarkers of sensitivity to Aurora kinase inhibitors have been identified. SCLC cells with MYC amplification or high gene expression frequently respond to Aurora B inhibitors, although an independent study demonstrated that neither MYC expression nor amplification is required for sensitivity to Aurora A kinase inhibition in RB1-mutant SCLC cells. However, in the squamous cancer cell lines examined in the present disclosure, Myc protein expression did not correlate with sensitivity to Aurora kinase inhibitors, which suggests that Rb1 loss is the more important driver of Mad2 expression. Other potential biomarkers of Aurora kinase inhibitor sensitivity in HNSCC are mutations in KMT2D (lysine methylraisferase 2D), although KMT2D mutations did not correlate with sensitivity in the current study, in which cell death, rather than inhibition of proliferation, was used as a metric of drug sensitivity. Previous studies have shown that methyltransferase regulates the SAC complex by directly binding to Mad2 and thereby limiting its availability. In HNSCC, mutations in KMT2D may create an imbalance of Mad2 and TRIP13 levels, which would make KMT2D-mutant cells dependent on Aurora kinases for their mitotic exit and survival.

To promote rapid translation of the methods and compositions provided by the present disclosure, alisertib was used for most of the studies because it is a specific inhibitor of Aurora A that is well-tolerated and under active clinical development. Alisertib has an established safety profile in human clinical trials. Common toxicities include anemia, thrombocytopenia, and neutropenia. Multiple clinical trials using Aurora kinase inhibition have not shown clinical efficacy of single agents in solid tumors. Indeed, even some HPV positive models experienced only minimal apoptosis with modest, clinically relevant concentrations of alisertib. However, the data demonstrate that the combination the TRIP13 and Aurora kinase inhibition will be effective in HPV positive cancers. The greatest unmet clinical need is for more effective systemic therapy for patients with recurrent/metastatic HNSCC that is resistant to cisplatin and/or radiotherapy. Several of the cell lines used are resistant to cisplatin and radiotherapy. In HNSCC patients who received 100 mg/m² cisplatin, the plasma Cₘₐₓ was 11 μM. Several HPV+ cell lines have IC₅₀ values above 11 μM including SiHa (28 μM), ME180 (12 μM), CaSki (15 μM), Vu147T (11.2 μM), UPCI-SCC-090 (11.1 μM) and UDSCC2 (18 μM). Additionally, SiHa and UPCI-SCCO90 are resistant to radiotherapy.

The data presented herein show that Aurora kinase and TRIP13 have synthetic lethality specifically in HPV-driven squamous cancer cells because the cells' loss of Rb protein expression leads to high Mad2 expression. HPV-positive tumors from patients with HNSCC also exhibited high levels of Mad2 expression. This cancer cell-specific effect allowed us to reduce the concentration of alisertib needed for treatment. This result demonstrates that the combination of TRIP13 and Aurora kinase inhibition provides a wider therapeutic window than classic cytotoxic agents do for the treatment of HPV-positive squamous cell carcinoma. This combination could help meet an urgent unmet need to reduce mortality and long-term treatment-related toxicity among patients with HPV-driven cancers. The findings may also be applicable to the treatment of patients with other cancers with Rb pathway defects, including NSCLC.

What is claimed is:

1. A method of treating HPV-positive cancer in a subject in need thereof, the method comprising administering a therapeutically effective amount of an Aurora kinase inhibitor to said subject.

2. The method of claim 1, wherein the HPV-positive cancer is selected from the group consisting of head and neck squamous cell carcinoma, cervical squamous cell carcinoma, anal squamous cell carcinoma, penile squamous cell carcinoma, and vulvar squamous cell carcinoma.

3. The method of claim 1, wherein the Aurora kinase inhibitor is selected from the group consisting of alisertib, CYC116, tozasertib, ZM447439, GSK1070916, PF03814735, SNS314, AMG900, MLN8054, CCT129202, CCT137690, MK5108, PHA680632, danusertib, TAK901, barasertib, ENMD-2076, AT-9283, KW-2449, ilorasertib, chiauranib, LY3295668 (AK-01), and TT-00420, or a combination thereof.

4. The method of claim 1, wherein the administering comprises local, regional, systemic, or continual administration.

5. The method of claim 1, wherein the administering comprises providing a single dose.

6. The method of claim 1, wherein the administering comprises providing multiple doses.

7. The method of claim 1, wherein the administering comprises oral, intravenous, or intramuscular administration.

8. The method of claim 1, wherein the administering comprises providing a pharmaceutical composition comprising the Aurora kinase inhibitor to the subject.

9. The method of claim 1, the method further comprising administering a second therapy to said subject.

10. The method of claim 9, wherein the second therapy is selected from the group consisting of an immunotherapy, a targeted cancer therapy, a chemotherapy, a radiation therapy, and surgery.

11. The method of claim 9, wherein administering the second therapy comprises administering a therapeutically effective amount of a therapeutic composition effective to decrease the activity of TRIP13 in the subject.

12. The method of claim 11, wherein the therapeutic composition comprises a protein, a peptide, a polypeptide, an RNA molecule, a peptidomimetic, an siRNA molecule, a gRNA molecule, or a small molecule, and wherein said therapeutic composition results in a reduction of TRIP13 activity in a cancer cell of the subject.

13. The method of claim 11, wherein said therapeutic composition performs a function selected from the group consisting of:
(a) reducing or inhibiting the expression of a gene encoding TRIP13;
(b) reducing or inhibiting the expression of a TRIP13 protein; and
(c) reducing or inhibiting the function of a TRIP13 protein.

14. The method of claim 11, wherein the therapeutic composition comprises an siRNA molecule complementary to at least a portion of an mRNA sequence encoded by the TRIP13 gene or a gRNA molecule complementary to at least a portion of the TRIP13 gene.

15. The method of claim 11, wherein the therapeutic composition comprises DCZ0415.

16. The method of claim 9, wherein the second therapy is administered prior to administering the Aurora kinase inhibitor, after administering the Aurora kinase inhibitor, or approximately simultaneously with the administration of the Aurora kinase inhibitor.

17. The method of claim 9, wherein administering the second therapy comprises local, regional, systemic, or continual administration.

18. The method of claim 9, wherein administering the second therapy comprises providing a single dose.

19. The method of claim 9, wherein administering the second therapy comprises providing multiple doses.

20. The method of claim 9, wherein administering the second therapy comprises oral, intravenous, or intramuscular administration.

21. A pharmaceutical composition comprising:
(a) an Aurora kinase inhibitor selected from the group consisting of alisertib, CYC116, tozasertib, ZM447439, GSK1070916, PF03814735, SNS314, AMG900, MLN8054, CCT129202, CCT137690, MK5108, PHA680632, danusertib, TAK901, barasertib, ENMD-2076, AT-9283, KW-2449, ilorasertib, chiauranib, LY3295668 (AK-01), and TT-00420; and
(b) DCZ0415, an siRNA molecule complementary to at least a portion of an mRNA sequence encoded by the TRIP13 gene, a gRNA molecule complementary to at least a portion of the TRIP13 gene, or a chemotherapeutic agent.

22. The pharmaceutical composition of claim 14, wherein the pharmaceutical composition further comprises a pharmaceutically acceptable carrier.

23. The pharmaceutical composition of claim 21, further comprising:
(a) at least two Aurora kinase inhibitors selected from the group consisting of alisertib, CYC116, tozasertib, ZM447439, GSK1070916, PF03814735, SNS314, AMG900, MLN8054, CCT129202, CCT137690, MK5108, PHA680632, danusertib, TAK901, barasertib, ENMD-2076, AT-9283, KW-2449, ilorasertib, chiauranib, LY3295668 (AK-01), and TT-00420; and
(b) at least two therapeutic molecules selected from the group consisting of DCZ0415, an siRNA molecule complementary to at least a portion of an mRNA sequence encoded by the TRIP13 gene, a gRNA molecule complementary to at least a portion of the TRIP13 gene, and a chemotherapeutic agent.
